(19) United States
(12) Patent Application Publication
(10) Pub. No.: US 2011/0262352 A1
(43) Pub. Date: Oct. 27, 2011

(54) ARYL PIPERAZINE AND THEIR USE AS ALPHA2C ANTAGONISTS

(76) Inventors: David Din Belle, Espoo (FI); Patrik Holm, Lielahti TL (FI); Arto Tolvanen, Espoo (FI); Gerd Wohlfahrt, Helsinki (FI)

(21) Appl. No.: 13/130,102

(22) PCT Filed: Nov. 20, 2009
(86) PCT No.: PCT/FI2009/000097

§ 371 Date: Jul. 7, 2011

Related U.S. Application Data
(60) Provisional application No. 61/193,355, filed on Nov. 20, 2008.

(51) Int. Cl. A61K 51/04; A61K 31/551; C07D 405/06; A61P 25/24; A61P 43/00; A61P 25/16; A61P 25/28; A61P 25/18; C07D 405/14; A61K 31/496

(52) U.S. Cl. 424/1.65; 540/575; 514/218; 544/376; 514/254.11

(57) ABSTRACT

Compounds of formula (I), wherein X, Z, A, B, D, E, R₁-R₄ and m are as defined in the claims, exhibit alpha2C antagonistic activity and are thus useful as alpha2 antagonists.

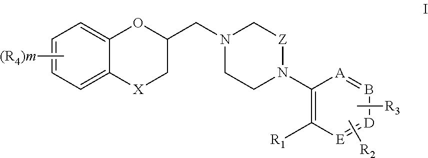

### FIELD OF THE INVENTION

The present invention relates to pharmacologically active aryl piperazines, or pharmaceutically acceptable salts and esters thereof, as well as to pharmaceutical compositions comprising them and to their use as alpha2C antagonists. The compounds of the invention can be used in their labeled or unlabeled form.

### BACKGROUND OF THE INVENTION

It is generally known and accepted in the art that compounds exhibiting alpha adrenergic activity may be used for the treatment of a wide variety of diseases and conditions of the peripheric system and the central nervous system (CNS).

The alpha adrenergic receptors can be divided on a pharmacological basis into alpha1 and alpha2 adrenoceptors, which can both be further divided into subtypes. Three genetically encoded subtypes, namely alpha2A, alpha2B, and alpha2C adrenoceptors, have been discovered in human. A fourth pharmacologically defined subtype, namely alpha2D adrenoceptor, is known in some other mammals and in rodents. It corresponds to the genetically defined alpha2A adrenoceptor.

The alpha2 adrenoceptor subtypes have distinct tissue distributions and functional roles. For instance, while alpha2A adrenoceptors are widely expressed in various tissues, alpha2C adrenoceptors are concentrated in the CNS and appear to play a role in the modulation of specific CNS mediated behavioral and physiological responses.

Some compounds that are non-specific for any of the above-mentioned alpha2 subtypes and some compounds that are specific for certain alpha2 subtypes are known in the art. For example, atipamezole disclosed in EP 183 492 A1 (compound XV at page 13) is a non-specific alpha2 antagonist. Compounds that are selective antagonists for the alpha2C subtype and are useful for the treatment of mental illness, e.g. mental disturbance induced by stress, are described in U.S. Pat. No. 5,902,807. Such compounds are, for example, MK-912 and BAM-1303. Imidazole derivatives having agonist-like activity for alpha2B or 2B/2C adrenoceptors are disclosed in WO 99/28300. Quinoline derivatives useful as alpha2 antagonists are disclosed in WO 01/64645 and WO 2004/067513. Arylquinolizine derivatives useful as alpha2 antagonists are disclosed in WO 03/082866.

In order to be able to reduce the risk of adverse events during treatment, an enhanced selectivity of the alpha2 antagonists, would be desirable. For example, the use of non-selective alpha2 antagonists is attributed with side effects, such as increases in blood pressure, heart rate, salivary secretion, gastrointestinal secretion, and anxiety. Also an enhanced potency of the alpha2C antagonists would be desirable, in order to be able to reduce the dose needed.

As to known aryl piperazines, 1-((2,3-dihydrobenzo[b][1,4]dioxin-2-yl)methyl)-4-(2-methoxyphenyl)piperazine has been disclosed in U.S. Pat. No. 3,362,956. 1-(Chroman-2-ylmethyl)-4-o-tolylpiperazine has been disclosed in Indian J. Chem. 20B (1981) 1063. A fluorophenyl piperazine has been disclosed for example, in Eur. J. Med. Chem. 35 (2000) 663.

### SUMMARY OF THE INVENTION

An object of the present invention is to provide further alpha2C antagonists that can be used for the treatment of diseases or conditions of the peripheric or central nervous system wherein alpha2C antagonists are indicated to be useful. Accordingly, an object of the present invention is to provide further compounds to be used as alpha2C antagonists in the treatment of mammals. Furthermore, pharmaceutical compositions comprising the present compounds are provided.

The alpha2 antagonists of the present invention have an improved selectivity for the alpha2C adrenoceptor subtype and/or an enhanced potency.

### DETAILED DESCRIPTION OF THE INVENTION

The present invention relates to novel alpha2C antagonists having the general formula I,

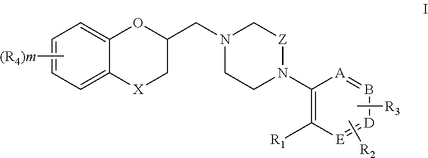

wherein

### X is O, S or CH₂;

Z is —[CH₂]ₙ—;
A, B, D and E are independently C or N provided that at least three of A, B, D and E are C;
R₁ is H, halogen, hydroxy, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, hydroxy(C₁-C₆)alkoxy(C₁-C₆)alkyl, (C₁-C₆)alkoxy(C₁-C₆)alkoxy(C₁-C₆)alkyl, (C₁-C₆)alkoxy-(C═O)—, CN, (R₅)₂N—, (R₅)₂N—(C₁-C₆)alkyl, (R₅)₂N—(C═O)—, SH—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, hydroxy(C₁-C₆)alkyl-S(Op)-(C₁-C₆)alkyl, (C₁-C₆)alkoxy(C₁-C₆)alkyl-S(Op)-(C₁-C₆)alkyl or furyl;
R₂ is H, halogen, (C₁-C₆)alkyl, (C₁-C₆)alkoxy or hydroxy(C₁-C₆)alkyl;
R₃ is H, halogen, (C₁-C₆)alkyl or phenyl;
R₄ is halogen, hydroxy, (C₁-C₆)alkyl, (C₁-C₆)alkoxy, CN or (R₅)₂N—;
R₅ is, independently at each occurence, H, (C₁-C₆)alkyl or (C₁-C₆)alkoxy(C₁-C₆)alkyl;
m is 0, 1 or 2;
n is 1 or 2; and
p is 1 or 2,
in labeled or unlabeled form, or a pharmaceutically acceptable salt or ester thereof,
with the provisos, that
a) R₁, R₂ and R₃ are not simultaneously H;
b) when A is C and two of R₁, R₂ and R₃ is H, then the third of R₁, R₂ and R₃ is not halogen;
c) the compound is not 1-((2,3-dihydrobenzo[b][1,4]dioxin-2-yl)methyl)-4-(2-methoxyphenyl)piperazine, 1-(chroman-2-ylmethyl)-4-o-tolylpiperazine or 1-((2,3-dihydrobenzo[b][1,4]dioxin-2-yl)methyl)-4-(6-methylpyridin-2-yl)piperazine.

In a possible subgroup of the compounds of formula I, X is O.

In a further possible subgroup of the compounds of formula I, A, B, D and E are C.

In another possible subgroup of the compounds of formula I, A is N; and B, D and E are C.

In a further possible subgroup of the compounds of formula I, n is 1.

In a further possible subgroup of the compounds of formula I, n is 2.

In another possible subgroup of the compounds of formula I,

### X is O, S or CH₂;

Z is —[CH₂]ₙ—;

### A is C or N;

### B, D and E are C;

R₁ is H, halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurence, H or (C₁-C₆)alkyl;
m is 0; and
n is 1 or 2; for example

### X is O;

Z is —[CH₂]ₙ—;

### A is C or N;

### B, D and E are C;

R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurence, H or (C₁-C₆)alkyl;
m is 0; and
n is 1 or 2; such as

### X is O;

Z is —[CH₂]ₙ—;

### A, B, D and E are C;

R₁ is (C₁-C₆)alkyl, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H or halogen;

### R₃ is H;

R₅ is, independently at each occurence, H or (C₁-C₆)alkyl;
m is 0; and
n is 1 or 2; or

### X is O;

Z is —[CH₂]ₙ—;

### A is N;

### B, D and E are C;

R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H or halogen;

### R₃ is H;

R₅ is, independently at each occurence, H or (C₁-C₆)alkyl;
m is 0; and
n is 1 or 2; or

### X is O;

Z is —[CH₂]ₙ—;

### A is N;

### B, D and E are C;

R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurence, H or (C₁-C₆)alkyl;
m is 0; and
n is 1; or

### X is O;

Z is —[CH₂]ₙ—;

### A is N;

### B, D and E are C;

R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurence, H or (C₁-C₆)alkyl;
m is 0; and
n is 2; or

### X is O;

Z is —[CH₂]ₙ—;

### A, B, D and E are C;

R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurence, H or (C₁-C₆)alkyl;
m is 0; and
n is 1; or

### X is O;

Z is —[CH₂]ₙ—;

### A, B, D and E are C;

R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurence, H or (C₁-C₆)alkyl;
m is 0; and
n is 2.

In yet another possible subgroup of the compounds of formula I, the compound is methyl 2-(4-((2,3-dihydrobenzo[b][1,4]dioxin-2-yl)methyl)piperazin-1-yl)benzoate, (2-(4-((2,3-dihydrobenzo[b][1,4]dioxin-2-yl)methyl)piperazin-1-yl)phenyl)methanol, 1-((2,3-dihydrobenzo[b][1,4]dioxin-2-yl)methyl)-4-(2-(methoxymethyl)phenyl)piperazine, 2-(4-((2,3-dihydrobenzo[b][1,4]dioxin-2-yl)methyl)piperazin-1-yl)benzonitrile, (2-(4-((2,3-dihydrobenzo[b][1,4]dioxin-2-yl)methyl)piperazin-1-yl)phenyl)methanamine, 1-(2-(4-((2,3-dihydrobenzo[b][1,4]dioxin-2-yl)methyl)piperazin-1-yl)phenyl)-N-methylmethanamine, 1-((2,3-dihydrobenzo[b][1,4]dioxin-2-yl)methyl)-4-(2-(ethoxymethyl)phenyl)piperazine, 2-(2-(4-((2,3-dihydrobenzo[b][1,4]dioxin-2-yl)methyl)piperazin-1-yl)phenyl)propan-2-ol, 1-((2,3-dihydrobenzo[b][1,4]dioxin-2-yl)methyl)-4-(3-(methoxymethyl)pyridin-2-yl)piperazine, (S)-(2-(4-((7-fluoro-2,3-dihydrobenzo[b][1,4]dioxin-2-yl)methyl)-piperazin-1-yl)pyridin-3-yl)methanol, (S)-(2-(4-((7-fluoro-2,3-dihydrobenzo[b][1,4]dioxin-2-yl)methyl)-piperazin-1-yl)pyridin-3-yl)methanol.HCl, (S)-1-((7-fluoro-2,3-dihydrobenzo[b][1,4]dioxin-2-yl)methyl)-4-(3-(methoxymethyl)pyridin-2-yl)piperazine .HCl, (S)-1-((2,3-dihydrobenzo[b][1,4]dioxin-2-yl)methyl)-4-(3-((2-fluoroethoxy)methyl)pyridin-2-yl)piperazine, 1-(2,3-dichlorophenyl)-4-((2,3-dihydrobenzo[b][1,4]dioxin-2-yl)methyl)piperazine, (2-(4-((2,3-dihydrobenzo[b][1,4]dioxin-2-yl)methyl)piperazin-1-yl)pyridin-3-yl)methanol, (S)-(2-(4-((2,3-dihydrobenzo[b][1,4]dioxin-2-yl)methyl)piperazin-1-yl)pyridin-3-yl)methanol, (S)-1-((2,3-dihydrobenzo[b][1,4]dioxin-2-yl)methyl)-4-(2-(methoxymethyl)phenyl)piperazine, (R)-1-((2,3-dihydrobenzo[b][1,4]dioxin-2-yl)methyl)-4-(2-(methoxymethyl)phenyl)piperazine, (S)-(2-(4-((2,3-dihydrobenzo[b][1,4]dioxin-2-yl)methyl)piperazin-1-yl)phenyl)methanol, (S)-1-((2,3-dihydrobenzo[b][1,4]dioxin-2-yl)methyl)-4-(3-(methoxymethyl)pyridin-2-yl)piperazine, (1-((2,3-dihydrobenzo[b][1,4]oxathiin-2-yl)methyl)-4-(2-(methoxymethyl)phenyl)piperazine, 1-(chroman-2-ylmethyl)-4-(2-(methoxymethyl)phenyl)piperazine, (2-(4-((2,3-dihydrobenzo[b][1,4]dioxin-2-yl)methyl)piperazin-1-yl)-6-fluorophenyl)methanol, (2-(4-((2,3-dihydrobenzo[b][1,4]dioxin-2-yl)methyl)piperazin-1-yl)-3-fluorophenyl)methanol, (2-(4-((2,3-dihydrobenzo[b][1,4]dioxin-2-yl)methyl)piperazin-1-yl)-5-fluorophenyl)methanol, (S)-1-((2,3-dihydrobenzo[b][1,4]dioxin-2-yl)methyl)-4-(2-propylphenyl)piperazine, (S)-1-((2,3-dihydrobenzo[b][1,4]dioxin-2-yl)methyl)-4-(2-(trifluoromethoxy)phenyl)piperazine, (S)-1-(biphenyl-3-yl)-4-((2,3-dihydrobenzo[b][1,4]dioxin-2-yl)methyl)piperazine, (S)-1-((2,3-dihydrobenzo[b][1,4]dioxin-2-yl)methyl)-4-(2-(furan-2-yl)phenyl)piperazine, (S)-ethyl 2-(4-((2,3-dihydrobenzo[b][1,4]dioxin-2-yl)methyl)piperazin-1-yl)benzoate, (S)-1-((2,3-dihydrobenzo[b][1,4]dioxin-2-yl)methyl)-4-o-tolylpiperazine, (S)-1-((2,3-dihydrobenzo[b][1,4]dioxin-2-yl)methyl)-4-m-tolylpiperazine, (S)-(3-(4-((2,3-dihydrobenzo[b][1,4]dioxin-2-yl)methyl)piperazin-1-yl)-4-methylphenyl)methanol, (S)-(3-(4-((2,3-dihydrobenzo[b][1,4]dioxin-2-yl)methyl)piperazin-1-yl)phenyl)methanol, (S)-2-(2-(4-((2,3-dihydrobenzo[b][1,4]dioxin-2-yl)methyl)piperazin-1-yl)phenyl)ethanol, methyl 2-(4-((2,3-dihydrobenzo[b][1,4]dioxin-2-yl)methyl)-1,4-diazepan-1-yl)benzoate, (2-(4-((2,3-dihydrobenzo[b][1,4]dioxin-2-yl)methyl)-1,4-diazepan-1-yl)phenyl)methanol, 2-(4-((2,3-dihydrobenzo[b][1,4]dioxin-2-yl)methyl)-1,4-diazepan-1-yl)nicotinonitrile, 2-(4-((2,3-dihydrobenzo[b][1,4]dioxin-2-yl)methyl)-1,4-diazepan-1-yl)nicotinamide, (2-(4-((2,3-dihydrobenzo[b][1,4]dioxin-2-yl)methyl)-1,4-diazepan-1-yl)pyridin-3-yl)methanol or (S)-(2-(4-((2,3-dihydrobenzo[b][1,4]dioxin-2-yl)methyl)-1,4-diazepan-1-yl)pyridin-3-yl)methanol.

The terms employed herein have the meanings indicated below. The term “at least one” employed in the meanings below refers to one or several, such as one.

The term “hydroxy”, as employed herein as such or as part of another group, refers to a —OH group.

The term “(C₁-C₆)alkyl”, as employed herein as such or as part of another group, refers to a straight or branched chain saturated hydrocarbon group having 1, 2, 3, 4, 5 or 6 carbon atom(s). Representative examples of (C₁-C₆)alkyl include, but are not limited to, methyl, ethyl, n-propyl, iso-propyl, n-butyl, iso-butyl, sec-butyl, test-butyl, n-pentyl, iso-pentyl, and n-hexyl.

The term “(C₁-C₆)alkoxy”, as employed herein as such or as part of another group, refers to an (C₁-C₆)alkyl group, as defined herein, appended to the parent molecular moiety through an oxygen atom. Representative examples of (C₁-C₆)alkoxy include, but are not limited to, methoxy, ethoxy, n-propoxy, n-butoxy, iso-butoxy, sec-butoxy, tert-butoxy, 2,2-dimethylpropoxy, 3-methylbutoxy, and n-hexoxy.

The term “halo” or “halogen”, as employed herein as such or as part of another group, refers to fluorine, chlorine, bromine or iodine.

The term “hydroxy(C₁-C₆)alkyl”, as employed herein as such or as part of another group, refers to at least one hydroxy group, as defined herein, appended to the parent molecular moiety through an (C₁-C₆)alkyl group, as defined herein. Representative examples of hydroxy(C₁-C₆)alkyl include, but are not limited to, hydroxymethyl, 1-hydroxyethyl, 2-hydroxyethyl, 2,2-dihydroxyethyl, 1-hydroxypropyl, 3-hydroxypropyl, 1-hydroxy-1-methylethyl, and 1-hydroxy-1-methylpropyl.

The term “(C₁-C₆)alkoxy(C₁-C₆)alkyl”, as employed herein as such or as part of another group, refers to at least one (C₁-C₆)alkoxy group, as defined herein, appended to the parent molecular moiety through an (C₁-C₆)alkyl group, as defined herein. When there are several (C₁-C₆)alkoxy groups, the (C₁-C₆)alkoxy groups can be identical or different. Representative examples of (C₁-C₆)alkoxy(C₁-C₆)alkyl include, but are not limited to, methoxymethyl, ethoxymethyl, propoxymethyl, 2-methoxyethyl, 2-ethoxyethyl, 2,2-dimethoxyethyl, 1-methyl-2-propoxyethyl, 1-methoxy-1-methylethyl, and 4-methoxybutyl.

The term “hydroxy(C₁-C₆)alkoxy”, as employed herein as such or as part of another group, refers to at least one hydroxy group, as defined herein, appended to the parent molecular moiety through an (C₁-C₆)alkoxy group, as defined herein. Representative examples of hydroxy(C₁-C₆)alkoxy include, but are not limited to, hydroxymethoxy, dihydroxymethoxy, 2-hydroxyethoxy, 2-hydroxypropoxy, 3-hydroxypropoxy, 2-hydroxybutoxy, and 2-hydroxy-1-methylethoxy.

The term “(C₁-C₆)alkoxy(C₁-C₆)alkoxy”, as employed herein as such or as part of another group, refers to at least one (C₁-C₆)alkoxy group, as defined herein, appended to the parent molecular moiety through an (C₁-C₆)alkoxy group, as defined herein. The (C₁-C₆)alkoxy groups can be identical or different. Representative examples of (C₁-C₆)alkoxy(C₁-C₆)alkoxy include, but are not limited to, methoxymethoxy, propoxymethoxy, 2-methoxyethoxy, 2-ethoxyethoxy, 2-butoxyethoxy, 2,2-dimethoxyethoxy, 1-methyl-2-propoxyethoxy, 2-methoxypropoxy and 4-methoxybutoxy.

The term “halo(C₁-C₆)alkoxy”, as employed herein as such or as part of another group, refers to at least one halogen, as defined herein, appended to the parent molecular moiety through an (C₁-C₆)alkoxy group, as defined herein. When there are several halogens, the halogens can be identical or different. Representative examples of halo(C₁-C₆)alkoxy include, but are not limited to, fluoromethoxy, chloromethoxy, difluoromethoxy, trifluoromethoxy, 2-bromoethoxy, 2,2,2-trichloroethoxy, 3-bromopropoxy, 2-chloropropoxy, and 4-chlorobutoxy.

The expression “compounds of the invention” as employed herein refers to the compounds of formula I.

Pharmaceutically acceptable salts, e.g. acid addition salts, with both organic and inorganic acids, are known in the field of pharmaceuticals. Representative examples of pharmaceutically acceptable acid addition salts include, but are not limited to, chlorides, bromides, sulfates, nitrates, phosphates, sulfonates, methane sulfonates, formates, tartrates, maleates, citrates, benzoates, salicylates, ascorbates, acetates and oxalates.

Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols. Representative examples of pharmaceutically acceptable esters include, but are not limited to, methyl, ethyl, n-propyl, iso-propyl, n-butyl, iso-butyl, sec-butyl, tert-butyl, and benzyl esters.

The invention includes within its scope all the possible geometric isomers, e.g. Z and E isomers (cis and trans isomers), of the compounds as well as all the possible optical isomers, e.g. diastereomers and enantiomers, of the compounds. Furthermore, the invention includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof, conventional resolution methods, e.g. fractional crystallization, may be used.

The invention further includes isotopically-labeled compounds of formula I; for example a carbon-isotope labeled compound of formula I; such as (S)-1-((2,3-dihydrobenzo[b][1,4]dioxin-2-yl)methyl)-4-(3-([¹¹C]-methoxymethyl)pyridin-2-yl)piperazine.

An isotopically or radio-labeled compound is a compound of formula I, wherein one or more atoms are replaced or substituted by an atom having an atomic mass or mass number different from the atomic mass or mass number typically found in nature. Examples of isotopes that can be incorporated into compounds of the invention include, but are not limited to, isotopes of hydrogen, carbon, nitrogen, oxygen, phosphorous, sulfur, fluorine, iodine and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, or any subset thereof. The radionuclide that is incorporated in the instant radio-labeled compounds will depend on the specific application of that radio-labeled compound. Positron emitting isotopes such as ¹¹C, ¹³N, ¹⁵O, and ¹⁸F are useful for positron emissing tomography (PET) studies. (Textbook of drug design and discovery. 3rd edition, Chapter 8: Radiotracers: synthesis and use in imaging by C. Halldin and T. Högberg.)

PET is so far the only method that can offer quantitative information on molecular recognition (e.g. receptor binding) in vivo in man. However, there have not been tracers available for studying alpha2C adrenoceptor occupancy. The labeled compounds of formula I can be used as novel alpha2C-receptor selective PET tracers in humans and animals; for example, carbon-11 labeled compounds of formula I be used as novel alpha2C-receptor selective PET tracers.

Isotopically labeled compounds of the invention can generally be prepared by following procedures analogous to those disclosed in the schemes and/or in the examples herein below, by substituting an isotopically labeled reagent for a non-isotopically labeled reagent. For example, carbon-isotope labeled compounds of formula I can be prepared by methylation of a suitable precursor, using several different ¹¹C-labeled methylating agents. Representative examples of ¹¹C-labeled methylating agents include, but are not limited to, ¹¹C-iodomethane, ¹¹C-bromomethane and ¹¹C-methyl triflate. A person skilled in the art also understands that said suitable precursor must contain a suitable reactive functional group, such as hydroxy, thiol, carboxyl or amino.

Compounds of the invention can be prepared by a variety of synthetic routes analogously or according to the methods known in the literature using suitable starting materials. The starting materials used in the processes herein are either commercially available or can be prepared via synthetic routes known in the literature.

Compounds of formula I are generally made up of a suitable acid and an aryl piperazine fragment. For example, the benzodioxane ring system containing starting materials are compounds of formulae A and B:

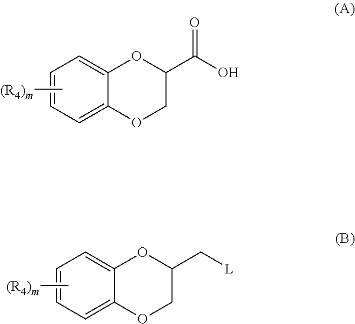

One starting compound is 2,3-dihydrobenzo[b][1,4]dioxine-2-carboxylic acid (formula A, R₄=H), which is commercially available and also easily resolved into its enantiomers as described in Tetrahedron: Asymmetry 16 (2005) 1639.

Compounds of formula B, possessing a leaving group L (most suitably a halogen, mesylate or tosylate) and group(s) R₄ (as defined above) can be prepared according to known methods. When R₄=H, enantiomers of formula B are easily derived from the corresponding enantiomer of formula A via reduction and insertion of a desired leaving group.

The other half in formula I, i.e. aryl piperazines and homopiperazines of formula C, when not commercially available, can be synthesized by alkylation of piperazine, in most cases N-protected, with an electron deficient aryl halide.

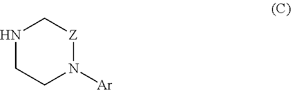

In formula C, Z is as defined above and Ar is:

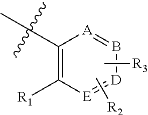

wherein A, B, D, E and R₁-R₃ are as defined above.

In general, compounds of formula I can be prepared analogously or according to the following scheme 1:

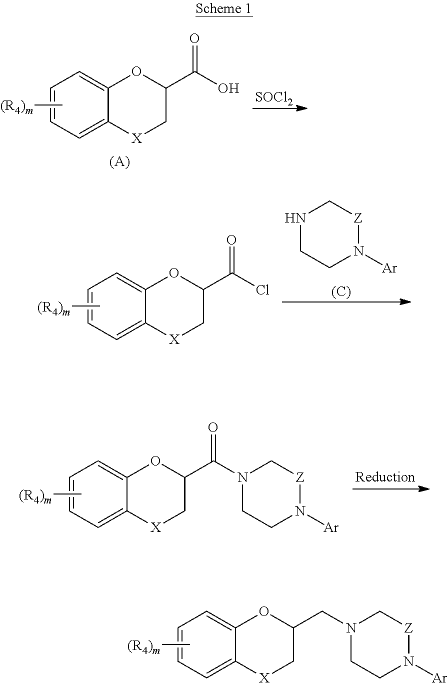

wherein X, Z, Ar, R₄ and m are as defined above. This method is especially suitable for preparing the enantiomers of formula I, as the enantiomers of formula A are easily available.

Another route for preparing compounds of formula I is the direct alkylation of aryl piperazines C with benzodioxanes B as shown in scheme 2:

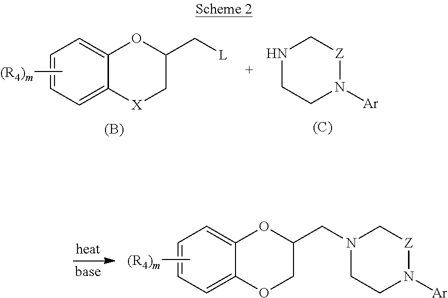

wherein L, R₄, m, Z and Ar are as defined above.

Furthermore, the common method to construct aryl piperazines via ring closure of bis(chloroethyl)amines with anilines (e.g. Tetrahedron Lett. 46 (2005) 7921 and references cited therein) is applied to benzodioxane derivatives. Reacting compound D with substituted anilines E gives rise to compounds of formula I (scheme 3):

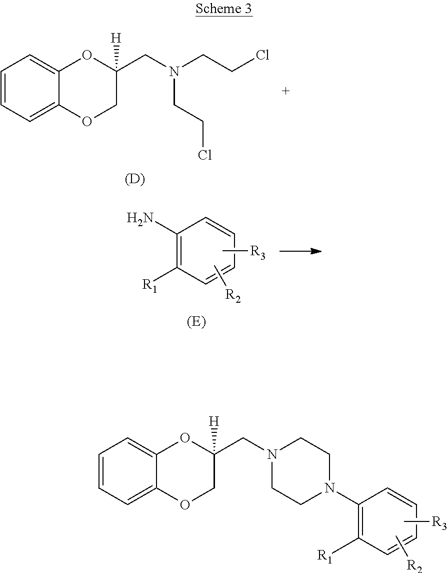

wherein R₁-R₃ are as defined above.

In some cases, as compared to scheme 2, a slightly modified order of events is employed. Commercial (2,3-dihydrobenzo[b][1,4]dioxin-2-yl)(piperazin-1-yl)methanone F is alkylated with electron deficient aryl halides G to afford intermediates H, which are further transformable to certain compounds of formula I (scheme 4):

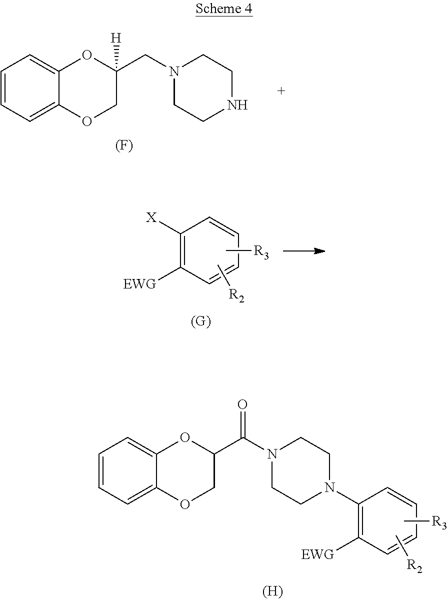

wherein X is halogen, EWG is an electron withdrawing group (e.g. COOR, CHO etc.) and R₂ and R₃ defined as above.

In the synthesis of homopiperazines, mainly the routes described in schemes 1 and 2 are utilized.

A person skilled in the art realizes that any starting material or intermediate in the reactions described above can be protected, if necessary, in a manner known in the art. Any protected functionality can subsequently be deprotected in a manner known in the art.

The synthetic routes described above are meant to illustrate the preparation of the compounds of formula I and the preparation is by no means limited thereto, i.e. there are also other possible synthetic methods which are within the general knowledge of a person skilled in the art.

The compounds of formula I may be converted, if desired, into their pharmaceutically acceptable salt or ester form using methods known in the art.

The present invention will be explained in more detail by the following examples. The examples are meant for illustrating purposes only and do not limit the scope of the invention defined in the claims.

Abbreviations: ACN=acetonitrile, DCM=dichloromethane, DIPEA=N,N-diisopropylethylamine, DMF=N,N-dimethylformamide, EtOAc=ethyl acetate, IPA=isopropanol, LAH=lithium aluminum hydride, LC-MS=liquid chromatography-mass spectrometry, RT=room temperature, THF=tetrahydrofuran, TLC=thin layer chromatography.

Column chromatography was performed on Silica gel 60 obtained from Merck, or using a CombiFlash instrument together with Redisep columns, both provided by Teledyne ISCO. Microwave heating was performed using an Emrys Optimiser microwave reactor from Personal Chemistry or an Initiator 2.0 microwave reactor from Biotage. The structures of the products were confirmed by ¹H NMR. The spectra were measured with a Bruker Avance 400 instrument. LC-MS analyses were performed using Waters 2690 Alliance HPLC and Waters Micromass ZQ4000 single quadrupole mass spectrometer using ESI.

### Preparation of Starting Materials

Methyl 2-(piperazin-1-yl)benzoate was prepared in two steps from 1-benzylpiperazine and methyl 2-fluorobenzoate following the lines in WO 03/009850.

Methyl 2-(1,4-diazepan-1-yl)nicotinate was prepared using the method described in U.S. Pat. No. 6,562,827.

(R)-2,3-Dihydrobenzo[b][1,4]dioxine-2-carboxylic acid was resolved from the commercially available racemate as described in Tetrahedron: Asymmetry 16 (2005) 1639.

(R)-(2,3-Dihydrobenzo[b][1,4]dioxin-2-yl)methyl methanesulfonate was prepared from (R)-2,3-dihydrobenzo[b][1,4]dioxine-2-carboxylic acid according to Tetrahedron: Asymmetry 14 (2003) 3779.

(R)-2,3-Dihydrobenzo[b][1,4]dioxine-2-carbonyl chloride was prepared in the standard manner by refluxing (R)-2,3-dihydrobenzo[1,4]dioxine-2-carboxylic acid with excess thionyl chloride in toluene for 3 h. Evaporation to dryness gave the acid chloride in high yield.

2,3-Dihydrobenzo[b][1,4]oxathiine-2-carboxylic acid was prepared from ethyl 2,3-dihydrobenzo[b][1,4]oxathiine-2-carboxylate according to J. Med. Chem. 27 (1984) 1535.

### Preparation of Intermediates

### Intermediate A1

### Ethyl 2-(4-benzylpiperazin-1-yl)nicotinate

A mixture of ethyl 2-chloronicotinate (43.5 g, 234 mmol), 1-benzylpiperazine (39.8 ml, 234 mmol) and K₂CO₃ (35.5 g, 257 mmol) in DMF (200 ml) was refluxed for 4 h. After cooling to RT the mixture was poured into ice water (800 ml). The water phase was extracted 3× with EtOAc and the combined organic layers were washed with water and brine. Drying and evaporation gave 75.5 g of the title compound.

¹H NMR (CDCl₃): δ 1.36 (t, 3H), 2.54-2.57 (m, 4H), 3.43-3.46 (m, 4H), 3.55 (s, 2H), 4.32 (q, 2H), 6.69-6.72 (dd, 1H), 7.34-7.36 (m, 5H), 7.92-7.96 (dd, 1H), 8.24-8.27 (dd, 1H).

### Intermediate A2

### (2-(4-Benzylpiperazin-1-yl)pyridin-3-yl)methanol

LAH pellets (9.3 g, 246 mmol) were dissolved in dry THF (240 ml) at 45° C. under nitrogen atmosphere. After cooling to RT ethyl 2-(4-benzylpiperazin-1-yl)nicotinate (40 g, 123 mmol) in dry THF (300 ml) was added. The mixture was refluxed for 2 h 15 min. 4 M KOH (61.5 ml) was slowly added to the hot reaction mixture and stirring was continued for additional 20 min at 60° C. The precipitate was filtered and washed with EtOAc and the filtrate was evaporated to dryness to give 33.6 g of the title alcohol.

¹H NMR (CDCl₃): δ 2.62-2.65 (m, 4H), 3.16-3.19 (m, 4H), 3.59 (s, 2H), 4.20-4.35 (br s, 1H), 4.73 (s, 2H), 6.97-7.00 (dd, 1H), 7.24-7.37 (m, 5H), 7.53-7.55 (dd, 1H), 8.26-8.28 (dd, 1H).

### Intermediate A3

### 1-Benzyl-4-(3-(methoxymethyl)pyridin-2-yl)piperazine

A dispersion of NaH (60% in oil, 14 g, 349 mmol) in dry THF (170 ml) was heated to 60° C. under nitrogen atmosphere. (2-(4-Benzylpiperazin-1-yl)pyridin-3-yl)methanol (33 g, 116 mmol) in dry THF (170 ml) was added dropwise to the mixture. After stirring for 3 h at 60° C., the mixture was cooled to 0° C. and methyl iodide (9.4 ml, 151 mmol) in dry THF (70 ml) was added. The mixture was further stirred for 1 h at RT and again cooled to 0° C. Water was added until foaming stopped and after that more water (300 ml) was added. The crude product was extracted with EtOAc and the combined organic layers were evaporated. Water (300 ml) was added to the residue and the pH was adjusted to 1-2 with concentrated HCl. The mixture was stirred at 40-50° C. for 1 h, after which EtOAc was added and the phases were separated. The organic phase was washed once with 1 M HCl (30 ml). The acidic water phases were combined, the pH adjusted to 10 with 5 M NaOH followed by extraction with EtOAc (3×). The combined organic phases were washed with water, dried and evaporated to give 26.6 g of the title compound.

¹H NMR (CDCl₃): δ 2.59-2.62 (m, 4H), 3.19-3.22 (m, 4H), 3.40 (s, 3H), 3.59 (s, 2H), 4.40 (s, 2H), 6.90-6.93 (dd, 1H), 7.26-7.37 (m, 5H), 7.65-7.68 (dd, 1H), 8.22-8.24 (dd, 1H).

### Intermediate A4

### 1-(3-(Methoxymethyl)pyridin-2-yl)piperazine

Under nitrogen flow, a 1 liter reaction flask was charged with 10% Pd/C (5.26 g, 20 w-%) and MeOH (400 ml). 1-Benzyl-4-(3-(methoxymethyl)pyridin-2-yl)piperazine (26.3 g, 88 mmol) in MeOH (100 ml) and ammonium formate (16.7 g, 265 mmol) were added to the mixture, which was then refluxed for 2 h 15 min. During this period paraformaldehyde accumulated inside the condenser. The Pd catalyst was filtered off through a celite pad, which was washed with DCM. The combined filtrates were evaporated and brine and DCM were added to the residue. Organic phase was separated, washed with saturated NaHCO₃, dried and evaporated to dryness to afford 16.5 g of the title piperazine.

¹H NMR (CDCl₃): δ 3.03-3.05 (m, 4H), 3.14-3.17 (m, 4H), 3.42 (s, 3H), 4.43 (s, 2H), 6.92-6.95 (dd, 1H), 7.68-7.70 (dd, 1H), 8.23-8.25 (dd, 1H).

### Intermediate A5

### Ethyl 2-(piperazin-1-yl)nicotinate

As above, a mixture of ethyl 2-(4-benzylpiperazin-1-yl)nicotinate (14.97 g, 46.0 mmol), ammonium formate (6.38 g, 101 mmol) and 10% Pd/C (3 g, 46.0 mmol) in methanol (150 ml) was refluxed for 2 h. After cooling, the mixture was filtered through Celite. The filtrate was concentrated in vacuo to afford 10.25 g of the title compound.

¹H NMR (CDCl₃): δ 1.39 (t, 3H), 2.95-2.99 (m, 4H), 3.35-3.40 (m, 4H), 4.36 (q, 2H), 6.72-6.74 (m, 1H), 7.94-7.99 (m, 1H), 8.26-8.30 (m, 1H).

### Intermediate A6

### 1-Benzyl-4-(2-(methoxymethyl)phenyl)piperazine

As in the preparation of intermediate A3, (2-(4-benzylpiperazin-1-yl)phenyl)methanol (30 g, 0.106 mol) was treated with NaH (60% in oil, 13 g, 0.325 mol) in dry. THF (365 ml) at 60° C. for 4 h. The mixture was then cooled to 10° C. and MeI (8.6 ml, 1.3 eq) in THF (96 ml) was added. After stirring at 20-22° C. for 1 h, the mixture was cooled to 10° C. and water was added dropwise until the effervescence ceased. More water (600 ml) was then added, followed by extraction with EtOAc (3×500 ml). After removing the mineral oil originating from NaH, 29 g of the title compound was obtained.

¹H NMR (CDCl₃): δ 2.61 (br s, 4H), 2.96 (t, 4H), 3.41 (s, 3H), 3.58 (s, 2H), 4.52 (s, 2H), 7.05-7.11 (m, 2H), 7.22-7.39 (m, 6H), 7.42 (dd, 1H).

### Intermediate A7

### 1-(2-(Methoxymethyl)phenyl)piperazine

As in the preparation of intermediate A4, a mixture of 1-benzyl-4-(2-(methoxymethyl)phenyl)-piperazine (14.0 g, 47.2 mmol), ammonium formate (9.38 g, 0.149 mol) and 10% Pd/C (2.3 g) in MeOH (260 ml) was refluxed for 1 h. The catalyst was filtered, MeOH was evaporated and water (300 ml) was added to the residue. The aqueous phase was extracted with EtOAc (3×100 ml) and the combined organic layers were washed with water and 1 M NaHCO₃. After drying and evaporation 7.1 g of the title compound was obtained.

¹H NMR (CDCl₃): δ 1.57 (br s, 1H), 2.85-2.93 (m, 4H), 2.99-3.06 (m, 4H), 3.43 (s, 3H), 4.54 (s, 2H), 7.05-7.13 (m, 2H), 7.27 (ddd, 1H), 7.44 (dd, 1H).

### Intermediate A8

### t-Butyl 4-((2,3-dihydrobenzo[b][1,4]dioxin-2-yl)methyl)-1,4-diazepane-1-carboxylate

A mixture of 2-(bromomethyl)-2,3-dihydrobenzo[b][1,4]dioxine (150 mg, 0.65 mmol), t-butyl 1,4-diazepane-1-carboxylate (130 mg, 0.65 mmol) and DIPEA (0.4 ml, 2.32 mmol) in DMF (1.5 ml) was heated in a microwave reactor at 160° C. for 10 min and then poured into water. The mixture was extracted with EtOAc (3×20 ml). The organic layer was dried and evaporated to dryness. The crude product was purified by flash chromatography (gradient of heptane and EtOAc) to give 93.4 mg of the title compound.

¹H NMR (DMSO-d₆): δ 1.39 (s, 9H), 1.71 (m, 2H), 2.72 (m, 6H), 3.35 (m, 4H), 3.96 (m, 1H), 4.27 (m, 2H), 6.82 (m, 4H).

### Intermediate A9

### 1-((2,3-Dihydrobenzo[b][1,4]dioxin-2-yl)methyl)-1,4-diazepane

A mixture of t-butyl 4-((2,3-dihydrobenzo[b][1,4]dioxin-2-yl)methyl)-1,4-diazepane-1-carboxylate (1.68 g, 4.82 mmol) and conc. HCl (2 ml) was stirred at room temperature for 3 h. The mixture was poured into ice water, basified to pH 9 and extracted with DCM (3×20 ml). The organic layer was dried and evaporated to give 0.96 g of the title compound.

¹H NMR (CDCl₃): δ 1.80 (m, 2H), 2.52 (s, 1H), 2.60-3.10 (m, 10H), 3.98 (m, 1H), 4.27 (m, 2H), 6.82 (m, 4H).

### Intermediate A10

### (R)—N,N-Bis(2-chloroethyl)-2,3-dihydrobenzo[b][1,4]dioxine-2-carboxamide

(R)-2,3-Dihydrobenzo[b][1,4]dioxine-2-carbonyl chloride (11.1 mmol) was reacted with bis(2-chloroethyl)amine.HCl (1.89 g, 10.6 mmol) in the presence of triethylamine (3.43 ml, 24.4 mmol) in DCM (20 ml). Aqueous work-up, including washes with 1 M NaOH and 1 M HCl, gave 5.72 g of the title compound.

¹H NMR (CDCl₃): δ 3.64-3.82 (m, 6H), 3.99-4.01 (m, 2H), 4.26-4.36 (m, 1H), 4.48-4.55 (m, 1H), 4.91-4.96 (m, 1H), 6.83-6.95 (m, 4H).

### Intermediate A11

### (S)-2-Chloro-N-(2-chloroethyl)-N-((2,3-dihydrobenzo[b][1,4]dioxin-2-yl)methyl)ethanamine.HCl

(R)—N,N-Bis(2-chloroethyl)-2,3-dihydrobenzo[b][1,4]dioxine-2-carboxamide (3.04 g, 9.99 mmol) was dissolved in THF (50 ml). 1 M BH₃.THF-complex (50 ml, 50 mmol) was added and the mixture refluxed for 2 h under an inert atmosphere. After cooling, 6 M HCl (20 ml) was added and the mixture was stirred at 65 C for 20 min. The mixture was cooled and it was made alkaline by adding solid KOH. Water (50 ml) was added. The mixture was extracted with EtOAc (3×50 ml), the organic layers were pooled, dried and evaporated to dryness. The crude product was purified by flash chromatography to give 1.98 g of the title compound.

¹H NMR (CDCl₃): δ 2.81-2.88 (m, 1H), 2.91-3.10 (m, 4H), 3.50-3.59 (m, 5H), 4.00-4.25 (m, 1H), 4.19-4.27 (m, 1H), 4.28-4.34 (m, 1H), 6.81-6.91 (m, 4H).

### Preparation of Compounds of the Invention

### Via alkylation of aryl piperazines with 2-(bromomethyl)-2,3-dihydrobenzo[b][1,4]dioxine

### Example 1

### Methyl 2-(4-((2,3-dihydrobenzo[b][1,4]dioxin-2-yl)methyl)piperazin-1-yl)benzoate

A mixture of 2-(bromomethyl)-2,3-dihydrobenzo[b][1,4]dioxine (0.53 g, 2.406 mmol), methyl 2-(piperazin-1-yl)benzoate (0.55 g, 2.406 mmol) and K₂CO₃ (0.366 g, 2.647 mmol) in DMF (20 ml) was heated in a microwave reactor at 150° C. for 2 h. Water was added to the cooled mixture, which was then extracted three times with EtOAc. The combined extracts were washed well with water and brine, dried and evaporated to give the crude product, which was purified by flash chromatography (gradient of heptane and EtOAc) to give 0.397 g of the title compound.

¹H NMR (CDCl₃): δ 2.62-2.82 (m, 6H), 3.05-3.13 (m, 4H), 3.89 (s, 3H), 4.02 (dd, 1H), 4.29-4.40 (m, 2H), 6.80-6.92 (m, 4H), 7.02 (dd, 1H), 7.04 (d, 1H), 7.41 (dd, 1H), 7.73 (d, 1H).

### Example 2

### (2-(4-((2,3-Dihydrobenzo[b][1,4]dioxin-2-yl)methyl)piperazin-1-yl)phenyl)methanol

Methyl 2-(4-((2,3-dihydrobenzo[b][1,4]dioxin-2-yl)methyl)piperazin-1-yl)benzoate (0.60 g, 1.629 mmol) was reduced with LAH (0.31 g, 8.145 mmol) in THF (50 ml). After 1 h at RT, the mixture was worked up with 2 M NaOH to give the crude product, which was purified by flash chromatography (gradient of heptane and EtOAc) to afford 0.476 g of the title compound:

¹H NMR (CDCl₃): δ 2.62-2.90 (m, 6H), 3.00-3.06 (m, 4H), 4.04 (dd, 1H), 4.30-4.38 (m, 2H), 4.80 (s, 2H), 5.26 (br s, 1H), 6.81-6.93 (m, 4H), 7.08-7.32 (m, 4H).

### Example 3

### 1-((2,3-Dihydrobenzo[b][1,4]dioxin-2-yl)methyl)-4-(2-(methoxymethyl)phenyl)piperazine

(2-(4-((2,3-Dihydrobenzo[b][1,4]dioxin-2-yl)methyl)piperazin-1-yl)phenyl)methanol (356 mg, 1.05 mmol) was treated first with NaH (60%, 126 mg, 3.14 mmol) in dry THF (2 ml) at 60° C. for 2 h. Then, methyl iodide (0.08 ml, 1.26 mmol) in dry THF (1 ml) was added to the cooled (ca. 10° C.) mixture and stirring was continued for 1 h at RT. The mixture was poured into ice water and extracted with EtOAc. The crude product obtained after drying and evaporation was purified by flash chromatography (gradient of heptane and EtOAc) to give 155 mg of the title compound.

¹H NMR (CDCl₃): δ 2.62-2.82 (m, 6H), 2.82-3.02 (m, 4H), 3.42 (s, 3H), 4.03 (dd, 1H), 4.31-4.39 (m, 2H), 4.53 (s, 2H), 6.81-6.93 (m, 4H), 7.06-7.12 (m, 2H), 7.27 (dd, 1H), 7.43 (d, 1H).

The compound obtained above (155 mg, 0.44 mmol) was dissolved in EtOAc (3 ml) and 1 M HCl/Et₂O (0.6 ml) was added. The precipitate was filtered, washed with a small amount of cold EtOAc and dried in vacuo at 30° C. to afford 153 mg of the HCl salt of the title compound.

¹H NMR (DMSO-d₆): δ 3.17-3.28 (m, 4H), 3.29-3.64 (m, 5H), 3.34 (s, 3H), 3.77 (br d, 1H), 4.09 (dd, 1H), 4.36 (dd, 1H), 4.47 (s, 2H), 5.00 (m, 1H), 6.86-7.00 (m, 4H), 7.10-7.17 (m, 2H), 7.32 (dd, 1H), 7.39 (d, 1H), 11.27 (br s, 1H).

### Example 4

### 2-(4-((2,3-Dihydrobenzo[b][1,4]dioxin-2-yl)methyl)piperazin-1-yl)benzonitrile

A mixture of 2-(bromomethyl)-2,3-dihydrobenzo[b][1,4]dioxine (0.624 g, 2.72 mmol), 1-(2-cyanophenyl)piperazine (0.510 g, 2.72 mmol) and K₂CO₃ (0.414 g, 3.00 mmol) in DMF (20 ml) was heated in a microwave reactor at 200° C. for 7 min. Water was added to the cooled mixture, which was extracted three times with EtOAc. The combined extracts were washed well with water and brine, dried and evaporated to give the crude product, which was purified by flash chromatography (gradient of heptane and EtOAc) to yield 0.42 g of the title compound.

¹H NMR (CDCl₃): δ 2.67 (dd, 1H), 2.71-2.88 (m, 5H), 3.20-3.30 (m, 4H), 4.03 (dd, 1H), 4.29-4.39 (m, 2H), 6.81-6.93 (m, 4H), 6.97-7.05 (m, 2H), 7.48 (dd, 1H), 7.56 (d, 1H).

### Example 5

### (2-(4-((2,3-Dihydrobenzo[b][1,4]dioxin-2-yl)methyl)piperazin-1-yl)phenyl)methanamine

2-(4-((2,3-Dihydrobenzo[b][1,4]dioxin-2-yl)methyl)piperazin-1-yl)benzonitrile (0.55 g, 1.640 mmol) was reduced with LAH (124 mg, 3.28 mmol) in refluxing THF (20 ml) (2 h). Work-up with 2 N NaOH gave the crude product, which was purified by flash chromatography (gradient of heptane and EtOAc) to give 0.285 g of the title compound.

¹H NMR (CDCl₃): δ 1.62 (br s, 2H), 2.61-2.85 (m, 6H), 2.91-3.02 (m, 4H), 3.89 (s, 2H), 4.03 (dd, 1H), 4.30-4.39 (m, 2H), 6.79-6.93 (m, 4H), 7.09 (dd, 1H), 7.13 (d, 1H), 7.24 (dd, 1H), 7.31 (d, 1H).

### Example 6

### 1-(2-(4-((2,3-Dihydrobenzo[b][1,4]dioxin-2-yl)methyl)piperazin-1-yl)phenyl)-N-methylmethanamine

(2-(4-((2,3-Dihydrobenzo[b][1,4]dioxin-2-yl)methyl)piperazin-1-yl)phenyl)methanamine (240 mg, 0.71 mmol) was stirred in DCM (5 ml) and triethylamine (0.15 ml, 1.06 mmol). The mixture was cooled to 0° C. followed by addition of ethyl chloroformate (0.10 ml) in dry DCM (1 ml). The cooling bath was removed and stirring was continued for 30 min. Water was then added, the DCM phase was separated and the aqueous layer extracted once with DCM. After drying procedures, 231 mg of the carbamate intermediate was obtained. This was immediately reduced with LAH (85 mg, 2.24 mmol) in refluxing THF (5 ml). Work-up with 2 N NaOH gave the crude product, which was purified by flash chromatography (gradient of heptane and EtOAc) to afford 91 mg of the title compound.

¹H NMR (CDCl₃): δ 2.30 (br s, 1H), 2.44 (s, 3H), 2.60-2.73 (m, 6H), 2.91-3.04 (m, 4H), 3.80 (s, 2H), 4.03 (dd, 1H), 4.30-4.39 (m, 2H), 6.80-6.93 (m, 4H), 7.07 (dd, 1H), 7.12 (d, 1H), 7.24 (dd, 1H), 7.31 (d, 1H).

### Example 7

### 1-((2,3-Dihydrobenzo[b][1,4]dioxin-2-yl)methyl)-4-(2-(ethoxymethyl)phenyl)piperazine

Analogously to example 3, (2-(4-((2,3-dihydrobenzo[b][1,4]dioxin-2-yl)methyl)piperazin-1-yl)phenyl)methanol (335 mg, 0.984 mmol) was treated with NaH (3 eq) followed by reaction with ethyl iodide (184 mg, 1.181 mmol). The crude product was purified twice by flash chromatography (gradient of heptane and EtOAc) to furnish 31 mg of the title compound.

¹H NMR (CDCl₃): δ 1.25 (t, 3H), 2.61-2.83 (m, 6H), 2.90-3.05 (m, 4H), 3.58 (q, 2H), 4.03 (dd, 1H), 4.30-4.40 (m, 2H), 4.57 (s, 2H), 6.80-6.93 (m, 4H), 7.07 (d, 1H), 7.09 (dd, 1H), 7.25 (dd, 1H), 7.45 (d, 1H).

### Example 8

### 2-(2-(4-((2,3-Dihydrobenzo[b][1,4]dioxin-2-yl)methyl)piperazin-1-yl)phenyl)propan-2-ol

Methyl 2-(4-((2,3-dihydrobenzo[b][1,4]dioxin-2-yl)methyl)piperazin-1-yl)benzoate (370 to mg, 1.0 mmol) was refluxed with 3 M MeMgCl/THF (1.5 ml) in dry THF (7 ml) for 3 h. The excess Grignard reagent was destroyed by careful addition of 1 M HCl, after which the mixture was made alkaline with 1 M NaOH. More water was added and the aqueous phase was extracted with EtOAc. Washing the combined extracts with water, drying and evaporation gave 380 mg of the crude alcohol product. The pure title compound was obtained by purification by flash chromatography (gradient of heptane and EtOAc).

¹H NMR (CDCl₃): δ 1.58 (s, 6H), 2.45-2.60 (m, 2H), 2.66 (dd, 1H), 2.77 (dd, 1H), 2.94-3.16 (m, 6H), 4.04 (dd, 1H), 4.30-4.38 (m, 2H), 6.81-6.92 (m, 4H), 7.19 (dd, 1H), 7.26 (dd, 1H), 7.30 (d, 1H), 7.36 (d, 1H), 9.12 (br s, 1H).

### Example 9

### 1-((2,3-Dihydrobenzo[b][1,4]dioxin-2-yl)methyl)-4-(3-(methoxymethyl)pyridin-2-yl)piperazine

A mixture of 2-(bromomethyl)-2,3-dihydrobenzo[b][1,4]dioxine (1.6 g, 6.98 mmol), 1-(3-(methoxymethyl)pyridin-2-yl)piperazine (1.3 g, 6.27 mmol), K₂CO₃ (0.87 g, 6.30 mmol) and KI (52 mg, 0.31 mmol) in DMF (35 ml) was heated at 120° C. for 4.5 h. Water was added to the cooled mixture, which was then extracted with EtOAc. The combined organic layers were extracted with 1 N HCl, the acid phase was made alkaline and extracted with EtOAc. Drying and evaporation gave 1.99 g of the crude product, which was recrystallised from IPA to afford 1.23 g of the title compound.

¹H NMR (CDCl₃): δ 2.63-2.82 (m, 6H), 3.21 (br t, 4H), 3.42 (s, 3H), 4.03 (dd, 1H), 4.32-4.39 (m, 2H), 4.42 (s, 2H), 6.80-6.92 (m, 4H), 6.93 (dd, 1H), 7.68 (dd, 1H), 8.24 (dd, 1H).

### Example 10

### (S)-(2-(4-((7-Fluoro-2,3-dihydrobenzo[b][1,4]dioxin-2-yl)methyl)-piperazin-1-yl)pyridin-3-yl)methanol

### Step A: (S)-Ethyl 2-(4-((7-fluoro-2,3-dihydrobenzo[b][1,4]dioxin-2-yl)methyl)-piperazin-1-yl)nicotinate

A mixture of (R)-(7-fluoro-2,3-dihydrobenzo[b][1,4]dioxin-2-yl)methyl 4-methylbenzene-sulfonate (1.23 g, 3.6 mmol), ethyl 2-(piperazin-1-yl)nicotinate (0.85 g, 3.6 mmol) and K₂CO₃ (0.55 g, 4.0 mmol) in acetonitrile (10 ml) was heated in a microwave reactor at 150° C. for 40 min. The solvent was evaporated and water (50 ml) was added. Extraction with EtOAc (3×30 ml) afforded after drying and evaporation a crude product mixture which was purified with flash chromatography (gradient of DCM and MeOH) to give 1.12 g of the title compound.

¹H NMR (DMSO-d₆): δ 1.30 (t, 3H), 2.52-2.62 (m, 6H), 3.32-3.34 (m, 4H), 3.97 (dd, 1H), 4.27 (q, 2H), 4.31 (m, 1H), 4.42 (m, 1H), 6.66 (m, 1H), 6.78 (dd, 1H), 6.82 (dd, 1H), 6.88 (dd, 1H), 7.90 (dd, 1H), 8.37 (dd, 1H).

### Step B: (S)-(2-(4-((7-Fluoro-2,3-dihydrobenzo[b][1,4]dioxin-2-yl)methyl)-piperazin-1-yl)pyridin-3-yl)methanol

(S)-Ethyl 2-(4-((7-fluoro-2,3-dihydrobenzo[b][1,4]dioxin-2-yl)methyl)-piperazin-1-yl)nicotinate (1.12 g, 2.80 mmol) was dissolved in THF (10 ml) and added dropwise to the cooled (0-5° C.) solution of LAH (0.53 g, 14.0 mmol) in THF (10 ml). The mixture was then allowed to warm up to ambient temperature and after 2 h stirring, water (10 ml) was cautiously added to the mixture. Celite was added and the solids filtered and washed with EtOAc. The combined filtrates were evaporated to dryness and coevaporated once with toluene. Toluene (20 ml) and 1 M HCl (40 ml) were added, the layers were separated and water extracted with toluene (20 ml). The water phase was made alkaline with NaOH and extracted with EtOAc (2×40 ml). The organic phase was dried and evaporated to give 0.85 g of the title compound.

¹H NMR (CDCl₃): δ 2.64-2.79 (m, 6H), 3.18-3.20 (m, 4H), 3.97-4.02 (m, 1H), 4.09 (m, 1H), 4.29-4.37 (m, 2H), 4.74 (m, 2H), 6.52-6.57 (m, 1H), 6.61-6.64 (m, 1H), 6.78-6.82 (m, 1H), 7.00-7.03 (m, 1H), 7.56-7.58 (m, 1H), 8.28-8.29 (m, 1H).

### Example 11

### (S)-(2-(4-((7-Fluoro-2,3-dihydrobenzo[b][1,4]dioxin-2-yl)methyl)-piperazin-1-yl)pyridin-3-yl)methanol.HCl

(S)-(2-(4-((7-Fluoro-2,3-dihydrobenzo[b][1,4]dioxin-2-yl)methyl)-piperazin-1-yl)pyridin-3-yl)methanol (0.76 g, 2.1 mmol) was dissolved in IPA (4 ml) with warming and 8% HCl/EtOAc (4 ml) was added. The precipitate was filtered to give 0.56 g of the title product.

¹H NMR (CDCl₃): δ 3.38-3.46 (m, 4H), 3.57-3.67 (m, 3H), 4.07-4.15 (m, 5H), 4.28-4.32 (m, 1H), 4.68 (m, 2H), 5.08 (m, 1H), 6.52-6.57 (m, 1H), 6.60-6.65 (m, 1H), 6.71-6.74 (m, 1H), 6.83-6.87 (m, 1H), 8.16-8.18 (m, 1H), 8.28-8.30 (m, 1H).

### Example 12

### (S)-1-((7-Fluoro-2,3-dihydrobenzo[b][1,4]dioxin-2-yl)methyl)-4-(3-(methoxymethyl)pyridin-2-yl)piperazine .HCl

(R)-(7-Fluoro-2,3-dihydrobenzo[b][1,4]dioxin-2-yl)methyl 4-methylbenzenesulfonate (1.353 g, 4 mmol), 1-(3-(methoxymethyl)pyridin-2-yl)piperazine (0.829 g, 4 mmol), potassium carbonate (0.608 g, 4.40 mmol) and acetonitrile (10 ml) were mixed and heated in a microwave reactor at 120° C. for 60 minutes. The mixture was evaporated, water (50 ml) was added. The aqueous mixture was extracted with EtOAc (3×20 ml). The organics were dried and evaporated to dryness. Flash chromatography (gradient of heptane/EtOAc) gave a pure product which was dissolved in 10% HCl/EtOH and evaporated to dryness. This procedure was repeated. 1.64 g of the title compound was obtained.

¹H NMR (DMSO-d₆): δ 2.49-2.51 (m, 6H), 3.08-3.28 (m, 4H), 3.33 (s, 3H), 3.96-4.00 (m, 1H), 4.29-4.33 (m, 1H), 4.37 (s, 2H), 4.40-4.47 (m, 2H), 6.64-6.68 (m, 1H), 6.78-6.81 (m, 1H), 6.87-6.90 (m, 1H), 6.98-7.00 (m, 1H), 7.67-7-69 (m, 1H), 8.18-8.20 (m, 1H).

### Example 13

### (S)-1-((2,3-Dihydrobenzo[b][1,4]dioxin-2-yl)methy)-4-(3-((2-fluoroethoxy)methyl)pyridin-2-yl)piperazine

### Step A: (S)-2-((2-(4-((2,3-Dihydrobenzo[b][1,4]dioxin-2-yl)methyl)piperazin-1-yl)pyridin-3-yl)methoxy)ethanol

To a mixture of (S)-1-((2,3-dihydrobenzo[b][1,4]dioxin-2-yl)methyl)-4-(2-(methoxymethyl)phenyl)piperazine (300 mg, 0.88 mmol) and 50% NaOH (75 mL) was added tetra-n-butyl ammonium bromide (28 mg, 0.088 mmol, 10 mol %) and the mixture was stirred for 15 min. 2-(3-Bromoethoxy)tetrahydro-2H-pyran (0.42 ml, 2.64 mmol, 300 mol %) was added slowly and the reaction mixture was warmed to 60° C. After 2 h brine (100 mL) was added and the mixture was extracted with toluene (2×50 mL+75 mL). The combined organic layers were washed with brine (50 mL), dried with Na2SO4, filtered and concentrated to dryness. The residue was dissolved in acetone (10 mL) and 1 M HCl was added until the pH was ˜3 (pH paper). After overnight stirring pH was adjusted to 1-2 and the mixture was stirred overnight. The mixture was neutralised with 50% NaOH (pH paper) and acetone was evaporated. DCM (20 mL) was added and the mixture was washed with water (10 mL), saturated NaHCO3 (5 mL) and brine (10 mL). The organic layer was dried with Na2SO4, filtered and concentrated in vacuo. Purification by column chromatography (EtOAc:heptane, 3:2-4:1-0:1, v/v) afforded 253 mg of the title compound.

### Step B: (S)-1-((2,3-Dihydrobenzo[b][1,4]dioxin-2-yl)methy)-4-(3-((2-fluoroethoxy)methyl)pyridin-2-yl)piperazine

(S)-2-((2-(4-((2,3-Dihydrobenzo[b][1,4]dioxin-2-yl)methyl)piperazin-1-yl)pyridin-3-yl)methoxy)ethanol (250 mg, 0.65 mmol) and dry DCM (8.8 mL) were charged under nitrogen into a dry 50 mL round-bottomed flask fitted with a magnetical stirrer and thermometer. The solution was cooled to ˜0° C. and DAST (127 μL, 0.97 mmol, 150 mol %) was added. The reaction mixture was allowed to warm to room temperature and after 2 h more DAST (42 μL, 0.32 mmol, 50 mol %) was added. The reaction mixture was stirred for 7.5 h in total. Saturated Na2CO3 solution (3.9 mL) was added at 0° C. The mixture was allowed to warm to room temperature and water (1.5 mL) was added. The layers were separated and the aqueous layer was extracted with DCM (2×4 mL). The combined organic layers were dried with Na2SO4, filtered and concentrated in vacuo. Purification by column chromatography (EtOAc:heptane, 1:1-1:0, v/v) afforded 155 mg of the title compound.

¹H NMR (MeOH-d₄): δ 2.70-2.81 (m, 6H), 3.19-3.22 (m, 4H), 3.66-3.70 (m, 1H), 3.85-98 (m, 1H), 3.98-4.01, (m, 1H), 4.28-4.46 (m, 3H), 4.56 (s, 2H), 4.66-4.70 (m, 1H), 6.76-7.84 (m, 4H), 7.04-7.06 (m, 1H), 7.78-7.82 (m, 1H), 8.15-8.18 (m, 1H)

### Via Reduction of Amide Intermediates

### Example 14

### 1-(2,3-Dichlorophenyl)-4-((2,3-dihydrobenzo[b][1,4]dioxin-2-yl)methyl)piperazine

### Step A: (4-(2,3-Dichlorophenyl)piperazin-1-yl)(2,3-dihydrobenzo[b][1,4]dioxin-2-yl)methanone

2,3-Dihydrobenzo[b][1,4]dioxine-2-carbonyl chloride (220 mg, 1.11 mmol) was reacted with 1-(2,3-dichlorophenyl)piperazine (257 mg, 1.11 mmol) and triethylamine (0.23 ml, 1.66 mmol) in DCM (3 ml) at 0° C. The stirred mixture was then allowed to reach RT. Water was added, the DCM layer separated and the aqueous phase extracted once with DCM. The combined organic layers were washed with water, dried and evaporated to give 330 mg of the crude amide, which was used without purification in the next step.

¹H NMR (CDCl₃): δ 2.98-3.18 (m, 4H), 3.67-3.83 (m, 2H), 3.91-4.04 (m, 2H), 4.36 (dd, 1H), 4.53 (dd, 1H), 4.88 (dd, 1H), 6.80-7.25 (m, 7H).

### Step B: 1-(2,3-Dichlorophenyl)-4-((2,3-dihydrobenzo[b][1,4]dioxin-2-yl)methyl)piperazine

(4-(2,3-Dichlorophenyl)piperazin-1-yl)(2,3-dihydrobenzo[b][1,4]dioxin-2-yl)methanone (328 mg, 0.834 mmol) was reduced with LAH (158 mg, 4.17 mmol) in refluxing THF (20 ml) (3 h). Work-up with 2 N NaOH gave the crude product, which was purified by flash chromatography (heptane/EtOAc/triethylamine, 7:3:0.5) to give 134 mg of the title compound.

¹H NMR (CDCl₃): δ 2.63-2.85 (m, 6H), 3.05-3.15 (m, 4H), 4.03 (dd, 1H), 4.30-4.40 (m, 2H), 6.80-7.02 (m, 5H), 7.12-7.18 (m, 2H).

### Example 15

### (2-(4-((2,3-Dihydrobenzo[b][1,4]dioxin-2-yl)methyl)piperazin-1-yl)pyridin-3-yl)methanol

### Step A: Ethyl 2-(4-(2,3-dihydrobenzo[b][1,4]dioxine-2-carbonyl)piperazin-1-yl)nicotinate

2,3-Dihydrobenzo[b][1,4]dioxine-2-carbonyl chloride (1.032 mmol) was stirred in 7:3 THF/water (10 ml). Ethyl 2-(piperazin-1-yl)nicotinate (220 mg, 0.935 mmol) was added at 0° C. The mixture was then stirred at RT for 4 h. THF was removed by evaporation and the remaining aqueous phase was extracted with DCM (20 ml). The organic phase was washed (water, 1 M HCl and 1 M Na₂CO₃), dried and evaporated to dryness to afford 255 mg of the title compound.

¹H NMR (CDCl₃): δ 1.39 (t, 3H), 3.44-3.78 (m, 4H), 3.86-3.40 (m, 4H), 4.31-4.39 (m, 3H), 4.48-4.53 (m, 1H), 4.84.4.90 (m, 1H), 6.79-6.97 (m, 5H), 7.95-7.99 (m, 1H), 8.29-8.33 (m, 1H).

### Step B: (2-(4-((2,3-Dihydrobenzo[b][1,4]dioxin-2-yl)methyl)piperazin-1-yl)pyridin-3-yl)methanol

Ethyl 2-(4-(2,3-dihydrobenzo[b][1,4]dioxine-2-carbonyl)piperazin-1-yl)nicotinate (255 mg, 0.642 mmol) was dissolved in THF (3 ml) and cooled on an ice-bath. LAH (77 mg, 2.03 mmol) was added and the mixture was stirred for 1 h while the temperature was allowed to reach r.t. The reaction was quenched with water. The mixture was filtered through Celite and evaporated to dryness. Flash chromatography afforded 43 mg of the title compound. ¹H NMR (CDCl₃): see example 16.

### Example 16

### (S)-(2-(4-((2,3-Dihydrobenzo[b][1,4]dioxin-2-yl)methyl)piperazin-1-yl)pyridin-3-yl)methanol

### Step A: (R)-Ethyl 2-(4-(2,3-dihydrobenzo[b][1,4]dioxine-2-carbonyl)piperazin-1-yl)nicotinate

(R)-2,3-Dihydrobenzo[b][1,4]dioxine-2-carbonyl chloride (10.3 g, 52.0 mmol) in THF (30 ml) was added dropwise to an ice-cold mixture of THF (100 ml), water (40 ml), ethyl 2-(piperazin-1-yl)nicotinate (10.2 g, 43.4 mmol) and K₂CO₃ (5.99 g, 43.4 mmol). The temperature was allowed to reach RT. The mixture was stirred for 17 h and concentrated in vacuo. Water (100 ml) was added. This mixture was extracted with EtOAc (2×250 ml). The organic layers were pooled, dried and evaporated to dryness to afford 14.7 g of the crude product. Part of this (13.7 g) was purified by flash chromatography to afford 10.7 g of the title compound.

¹H NMR (CDCl₃): δ 1.39 (t, 3H), 3.44-3.78 (m, 4H), 3.86-3.40 (m, 4H), 4.31-4.39 (m, 3H), 4.48-4.53 (m, 1H), 4.84.4.90 (m, 1H), 6.79-6.97 (m, 5H), 7.95-7.99 (m, 1H), 8.29-8.33 (m, 1H).

### Step B: (S)-(2-(4-((2,3-Dihydrobenzo[b][1,4]dioxin-2-yl)methyl)piperazin-1-yl)pyridin-3-yl)methanol

To an ice-cold suspension of LAH (1.232 g, 30.8 mmol) and THF (120 ml) was added dropwise a solution of (R)-ethyl 2-(4-(2,3-dihydrobenzo[b][1,4]dioxine-2-carbonyl)piperazin-1-yl)nicotinate (3.065 g, 7.71 mmol) in THF (30 ml). The temperature was allowed to reach RT and the mixture was stirred for 3 h. Water (20 ml) was added. The mixture was filtered through Celite and evaporated to dryness. Flash chromatography gave 560 mg of the title compound.

¹H NMR (CDCl₃): δ 2.6-2.9 (m, 6H), 3.19 (br t, 4H), 4.03 (dd, 1H), 4.14 (br s, 1H), 4.30-4.39 (m, 2H), 4.73 (s, 2H), 6.80-6.92 (m, 4H), 7.00 (dd, 1H), 7.57 (dd, 1H), 8.27 (dd, 1H).

### Example 17

### (S)-1-((2,3-Dihydrobenzo[b][1,4]dioxin-2-yl)methy)-4-(2-(methoxymethyl)phenyl)piperazine

### Step A: (R)-(2,3-Dihydrobenzo[b][1,4]dioxin-2-yl)(4-(2-(methoxymethyl)phenyl)piperazin-1-yl)methanone

As in example 14, Step A, (R)-2,3-dihydrobenzo[b][1,4]dioxine-2-carbonyl chloride (220 mg, 1.11 mmol) was reacted with 1-(2-(methoxymethyl)phenyl)piperazine (229 mg, 1.11 mmol) and triethylamine (0.23 ml, 1.66 mmol) in DCM (3.3 ml) at 0° C. to give 300 mg of the crude desired amide.

¹H NMR (CDCl₃): δ 2.88-3.12 (m, 4H), 3.44 (s, 3H), 3.66-3.81 (m, 2H), 3.83-3.98 (m, 2H), 4.36 (dd, 1H), 4.52 (dd, 1H), 4.56 (s, 2H), 4.88 (dd, 1H), 6.82-6.95 (m, 4H), 7.08 (d, 1H), 7.14 (dd, 1H), 7.30 (dd, 1H), 7.45 (d, 1H).

### Step B: (S)-1-((2,3-Dihydrobenzo[b][1,4]dioxin-2-yl)methyl)-4-(2-(methoxymethyl)phenyl)piperazine

(R)-(2,3-Dihydrobenzo[b][1,4]dioxin-2-yl)(4-(2-(methoxymethyl)phenyl)-piperazin-1-yl)methanone (366 mg, 0.99 mmol) was reduced with LAH (188 mg, 4.97 mmol) in dry THF (22 ml, reflux 2 h). Work-up with 2 N NaOH gave the crude product, which was purified by flash chromatography (gradient of heptane and EtOAc) to give 90 mg of the title compound.

¹H NMR (CDCl₃): see example 3.

### Example 18

### (R)-1-((2,3-Dihydrobenzo[b][1,4]dioxin-2-yl)methyl)-4-(2-(methoxymethyl)phenyl)piperazine

The corresponding (R)-isomer was prepared analogously to the above example 17 by first reacting (S)-2,3-dihydrobenzo[b][1,4]dioxine-2-carbonyl chloride (220 mg, 1.11 mmol) and 1-(2-(methoxymethyl)phenyl)piperazine (229 mg, 1.11 mmol) in the presence of triethylamine (0.23 mL, 1.66 mmol) in DCM (3.3 mL). Reduction of the crude amide (345 mg, 0.94 mmol) with 5 eq of LAH (178 mg, 4.68 mmol) in refluxing THF gave, after purification by flash chromatography (gradient of heptane and EtOAc), 87 mg of the title compound.

¹H NMR (CDCl₃): see example 3.

### Example 19

### (S)-(2-(4-((2,3-Dihydrobenzo[b][1,4]dioxin-2-yl)methyl)piperazin-1-yl)phenyl)methanol

### Step A: (R)-Methyl 2-(4-(2,3-dihydrobenzo[b][1,4]dioxine-2-carbonyl)piperazin-1-yl)benzoate

As in example 17, (R)-2,3-dihydrobenzo[b][1,4]dioxine-2-carbonyl chloride (0.50 g, 2.54 mmol) was reacted with methyl 2-(piperazin-1-yl)benzoate (0.56 g, 2.54 mmol) and triethylamine (0.54 ml, 3.81 mmol) in DCM (8 ml) at 0° C. to give 0.87 g of the crude amide.

¹H NMR (CDCl₃): δ 3.00-3.23 (m, 4H), 3.68-3.83 (m, 2H), 3.88-4.00 (m, 2H), 3.90 (s, 3H), 4.35 (dd, 1H), 4.52 (dd, 1H), 4.87 (dd, 1H), 6.82-6.95 (m, 4H), 7.04-7.11 (m, 2H), 7.46 (dd, 1H), 7.81 (d, 1H).

### Step B: (S)-(2-(4-((2,3-Dihydrobenzo[b][1,4]dioxin-2-yl)methyl)piperazin-1-yl)phenyl)methanol

The above amide (0.87 g, 2.27 mmol) was reduced with LAH (0.52 g, 13.65 mmol) in dry THF (55 ml, reflux 2 h). Work-up with 2.5 M NaOH gave the crude product, which was purified by flash chromatography (gradient of heptane and EtOAc) to give 188 mg of the title compound.

¹H NMR (CDCl₃): see example 2.

### Example 20

### (S)-1-((2,3-Dihydrobenzo[b][1,4]dioxin-2-yl)methyl)-4-(3-(methoxymethyl)pyridin-2-yl)piperazine

### Step A: (R)-(2,3-Dihydrobenzo[b][1,4]dioxin-2-yl)(4-(3-(methoxymethyl)pyridin-2-yl)piperazin-1-yl)methanone

1-(3-(Methoxymethyl)pyridin-2-yl)piperazine (25 g, 121 mmol) and K₂CO₃ (25 g, 181 mmol) were dissolved in a mixture of water (200 ml) and THF (300 ml). Crude (R)-2,3-dihydrobenzo[b][1,4]dioxine-2-carbonyl chloride, previously prepared from (R)-2,3-dihydrobenzo[b][1,4]dioxine-2-carboxylic acid (28.3 g, 157 mmol), was dissolved in dry THF (100 ml) and added in 10 min to the mixture at 20±5° C. with efficient stirring. Stirring was continued for additional 30 min at RT, after which the phases were separated. The organic phase was washed with brine, dried and evaporated to afford 42.4 g of the title product.

¹H NMR (CDCl₃): δ 3.17-3.37 (m, 4H), 3.45 (s, 3H), 3.70-338 (m, 3H), 3.85-3.96 (m, 2H), 4.33-4.38 (m, 1H), 4.45 (s, 2H), 4.51-4.54 (m, 1H), 4.87-4.89 (dd, 1H), 6.85-7.02 (m, 5H), 7.71-7.73 (dd, 1H), 8.26-8.27 (dd, 1H).

### Step B: (S)-1-((2,3-Dihydrobenzo[b][1,4]dioxin-2-yl)methyl)-4-(3-(methoxymethyl)pyridin-2-yl)piperazine

(R)-(2,3-Dihydrobenzo[b][1,4]dioxin-2-yl)(4-(3-(methoxymethyl)pyridin-2-yl)piperazin-1-yl)-methanone (42 g, 108 mmol) was dissolved in THF (420 ml). 1 M BH₃.THF solution (397 ml, 397 mmol) was added slowly to the stirred solution while the temperature was kept under 40° C. Stirring was continued for 2.5 h at 40° C. After cooling to RT MeOH (120 ml) and water (65 ml) were added and the solvents were evaporated. To the residue were added EtOH (65 ml), water (65 ml) and conc. HCl (63 ml) and the mixture was heated for 1.5 h at 60° C. Under cooling the pH of the mixture was adjusted to 10 with 50% NaOH solution. DCM was added and the formed precipitate was filtered off. Phases were separated and the water phase was washed with DCM. Combined organic layers were dried and evaporated. The crude product was recrystallised from IPA to give 29 g of the pure title compound.

¹H NMR (CDCl₃): δ 2.65-2.79 (m, 6H), 3.20-3.22 (m, 4H), 3.42 (s, 3H), 4.01-4.06 (dd, 1H), 4.33-4.37 (m, 2H), 4.42 (s, 2H), 6.83-6.96 (m, 5H), 7.68-7.70 (dd, 1H), 8.23-8.25 (dd, 1H).

### Example 21

### (1-((2,3-Dihydrobenzo[b][1,4]oxathiin-2-yl)methyl)-4-(2-(methoxymethyl)phenyl)piperazine

### Step A: (2,3-Dihydrobenzo[b][1,4]oxathiin-2-yl)(4-(2-(methoxymethyl)phenyl)piperazin-1-yl)methanone

2,3-Dihydrobenzo[b][1,4]oxathiine-2-carboxylic acid (112 mg, 0.48 mmol) was treated with thionyl chloride (0.21 ml, 2.85 mmol) in refluxing toluene for 1 h. After cooling, the mixture was evaporated to dryness and redissolved in DCM (2 ml). This solution was added to a stirred mixture of 1-(3-(methoxymethyl)pyridin-2-yl)piperazine (100 mg, 0.48 mmol), triethylamine (0.080 ml, 0.57 mmol) and DCM (1 ml). After 30 min at RT, the mixture was washed with 1 M Na₂CO₃ and evaporated to dryness to give 119 mg of the crude amide.

¹H NMR (CDCl₃): δ 2.89-3.11 (m, 4H), 3.18-3.23 (m, 1H), 3.41-3.55 (m, 4H), 3.64-3.99 (m, 4H), 4.56 (s, 2H), 4.89-4.92 (m, 1H), 6.85-6.92 (m, 2H), 6.97-7.05 (m, 1H), 7.06-7.18 (m, 3H), 7.26-7.33 (m, 1H), 7.42-7.49 (m, 1H).

### Step B: (1-((2,3-Dihydrobenzo[b][1,4]oxathiin-2-yl)methyl)-4-(2-(methoxymethyl)phenyl)piperazine

The amide from step A (119 mg, 0.309 mmol) was reduced with LAH (65 mg, 1.70 mmol) in dry THF (4 ml), first at RT for 2 h and then at refluxing temperature for 30 min. Work-up with 1 M NaOH and water, gave the crude product after filtration and evaporation. This was purified by flash chromatography (gradient of heptane and EtOAc) to give 30 mg of the title compound.

¹H NMR (CDCl₃): δ 2.63-2.8.8 (m, 6H), 2.94-3.00 (m, 4H), 3.02-3.10 (m. 1H), 3.15-3.22 (m, 1H), 3.42 (s, 3H), 4.35-4.45 (m, 1H), 4.53 (s, 2H), 6.82-6.88 (m, 2H), 6.97-7.02 (m, 1H), 7.04-7.12 (m, 3H), 7.24-7.30 (m, 1H), 7.42-7.45 (m, 1H).

### Example 22

### 1-(Chroman-2-ylmethyl)-4-(2-(methoxymethyl)phenyl)piperazine

### Step A: Chroman-2-yl(4-(2-(methoxymethyl)phenyl)piperazin-1-yl)methanone

As in the above example, crude chroman-2-carbonyl chloride, prepared from chroman-2-carboxylic acid (198 mg, 1.11 mmol), was reacted with 1-(2-(methoxymethyl)phenyl)piperazine (229 mg, 1.11 mmol) in the presence of triethylamine (0.23 ml, 1.67 mmol) in DCM (3.3 ml) to 343 mg of the crude amide, which was used as such in the next step.

¹H NMR (CDCl₃): δ 2.20-2.29 (m, 2H), 2.82-3.08 (m, 6H), 3.43 (s, 3H), 3.68-3.95 (m, 4H), 4.56 (s, 2H), 4.82 (dd, 1H), 6.83-6.91 (m, 2H), 7.05-7.17 (m, 2H), 7.29 (ddd, 1H), 7.45 (dd, 1H).

### Step B: 1-(Chroman-2-ylmethyl)-4-(2-(methoxymethyl)phenyl)piperazine

The above amide (339 mg, 0.925 mmol) was reduced with LAH (176 mg, 4.63 mmol) in refluxing THF (20 ml) (3 h). Work-up with 2 N NaOH gave the crude product, which was purified by flash chromatography (gradient of heptane and EtOAc) to give 139 mg of the title compound.

¹H NMR (CDCl₃): δ 1.75-1.88 (m, 1H), 2.04-2.14 (m, 1H), 2.61-2.93 (m, 8H), 2.98 (br t, 4H), 3.42 (s, 3H), 4.20-4.28 (m, 1H), 4.54 (s, 2H), 6.80-6.87 (m, 2H), 7.02-7.12 (m, 4H), 7.26 (ddd, 1H), 7.43 (dd, 1H).

The compound was treated with 1 M HCl/Et₂O in EtOAc to form a HCl salt in the usual manner.

¹H NMR (DMSO-d₆): δ 1.68-1.82 (m, 1H), 2.01-2.11 (m, 1H), 2.72-2.93 (m, 2H), 3.16-3.28 (m, 4H), 3.30-3.65 (m, 8H), 3.72-3.81 (m, 1H), 4.48 (m, 2H), 4.65-4.75 (m, 1H), 6:83-6.92 (m, 2H), 7.08-7.18 (m, 4H), 7.32 (dd, 1H), 7.40 (d, 1H), 10.75 (br s, 1H).

Via Alkylation of Piperazine Derivatives with Electron-Deficient Haloarenes

### Example 23

### (2-(4-((2,3-Dihydrobenzo[b][1,4]dioxin-2-yl)methyl)piperazin-1-yl)-6-fluorophenyl)methanol

### Step A: 2-(4-(2,3-Dihydrobenzo[b][1,4]dioxine-2-carbonyl)piperazin-1-yl)-6-fluorobenzaldehyde

A mixture of (2,3-dihydrobenzo[b][1,4]dioxin-2-yl)(piperazin-1-yl)methanone (0.20 g, 0.81 mmol), 2,6-difluorobenzaldehyde (0.36 g, 2.56 mmol) and K₂CO₃ (0.59 g, 4.26 mmol) in DMF (7 ml) was heated in a microwave reactor at 160° C. for 20 min. The mixture was poured into water and extracted with EtOAc (3×5 ml). The organic layer was dried and evaporated to give 0.35 g of the title aldehyde.

¹H NMR (DMSO-d₆): δ 3.11 (m, 4H), 3.71 (m, 4H), 4.21 (m, 1H), 4.41 (m, 1H), 5.27 (m, 1H), 6.83 (m, 3H), 6.94 (m, 2H), 7.05 (m, 1H), 7.61 (m, 1H), 10.21 (s, 1H).

### Step B: (2-(4-((2,3-Dihydrobenzo[b][1,4]dioxin-2-yl)methyl)piperazin-1-yl)-6-fluorophenyl)methanol

The crude product from the above step (0.32 g, 0.90 mmol) in THF (5 ml) was added to a suspension of LAH (0.17 g, 4.46 mmol) in dry THF (2 ml). The reaction mixture was heated in a microwave reactor at 80° C. for 10 min, after which it was poured into ice water and extracted with EtOAc (3×10 ml). The combined organic layers were dried and evaporated. The crude product was purified by flash chromatography (gradient of heptane and EtOAc) to give 36 mg of the title compound.

¹H NMR (DMSO-d₆): δ 2.61 (m, 6H), 3.01 (m, 4H), 4.02 (m, 1H), 4.30 (m, 2H), 4.51 (s, 2H), 5.01 (s, 1H), 6.86 (m, 6H), 7.31 (M, 1H).

### Example 24

### (2-(4-((2,3-Dihydrobenzo[b][1,4]dioxin-2-yl)methyl)piperazin-1-yl)-3-fluorophenyl)methanol

### Step A: 2-(4-(2,3-Dihydrobenzo[b][1,4]dioxine-2-carbonyl)piperazin-1-yl)-3-fluorobenzaldehyde

(2,3-Dihydrobenzo[b][1,4]dioxin-2-yl)(piperazin-1-yl)methanone (0.20 g, 0.81 mmol), 2,3-difluorobenzaldehyde (0.18 g, 1.28 mmol) and K₂CO₃ (0.29 g, 2.13 mmol) in DMF (3 ml) were heated in a microwave reactor at 160° C. for 20 min. The mixture was poured into water and extracted with EtOAc (3×5 ml). After drying and evaporation, 0.14 g of the crude aldehyde was obtained.

¹H NMR (DMSO-d₆): δ 3.19 (m, 4H), 3.60 (m, 4H), 4.12 (m, 1H), 4.39 (m, 1H), 5.22 (m, 1H), 6.83 (m, 4H), 7.56 (m, 1H), 7.70 (m, 1H), 7.80 (m, 1H), 10.21 (s, 1H).

### Step B: (2-(4-((2,3-Dihydrobenzo[b][1,4]dioxin-2-yl)methyl)piperazin-1-yl)-3-fluorophenyl)methanol

The product obtained in the above manner (0.32 g, 0.90 mmol) in dry THF (4 ml) was added to a suspension of LAH (0.17 g, 4.46 mmol) in dry THF (2 ml). The reaction mixture was heated under microwaves at 80° C. for 10 min, after which it was worked up as in example 23, Step B. The crude product was purified by column chromatography (gradient of heptane and EtOAc) to give 18.0 mg of the title compound.

¹H NMR (DMSO-d₆): δ 2.61 (m, 6H), 3.01 (m, 4H), 4.02 (m, 1H), 4.30 (m, 2H), 4.53 (s, 2H), 5.01 (s, 1H), 6.85 (m, 6H), 7.30 (m, 1H).

### Example 25

### (2-(4-((2,3-Dihydrobenzo[b][1,4]dioxin-2-yl)methyl)piperazin-1-yl)-5-fluorophenyl)methanol

### Step A: 2-(4-(2,3-Dihydrobenzo[b][1,4]dioxine-2-carbonyl)piperazin-1-yl)-5-fluorobenzaldehyde

As in the two previous examples, (2,3-dihydrobenzo[b][1,4]dioxin-2-yl)(piperazin-1-yl)methanone (0.20 g, 0.81 mmol), 2,5-difluorobenzaldehyde (0.38 g, 2.70 mmol) and K₂CO₃ (0.62 g, 4.50 mmol) in DMF (7 ml) were reacted under microwaves at 160° C. for 15 min. Work-up as above gave 0.27 g of the aldehyde intermediate.

¹H NMR (DMSO-d₆): δ 3.10 (m, 4H), 3.70 (m, 4H), 4.21 (m, 1H), 4.40 (m, 1H), 5.28 (m, 1H), 6.83 (m, 3H), 6.90 (m, 2H), 7.40 (m, 1H), 7.60 (m, 1H), 10.18 (s, 1H).

### Step B: (2-(4-((2,3-Dihydrobenzo[b][1,4]dioxin-2-yl)methyl)piperazin-1-yl)-5-fluorophenyl)methanol

The product obtained in Step A (0.27 g, 0.70 mmol) was reduced with LAH (0.13 g, 3.50 mmol) in dry THF (5 ml) as above. The crude product was purified by flash chromatography (gradient of heptane and EtOAc) to give 13.1 mg of the title compound.

¹H NMR (DMSO-d₆): δ 2.61 (m, 6H), 3.01 (m, 4H), 4.03 (m, 1H), 4.51 (m, 2H), 4.51 (d, 2H), 5.45 (t, 1H), 6.86 (m, 6H), 7.21 (m, 1H).

### Aryl Piperazines Via Ring Closure

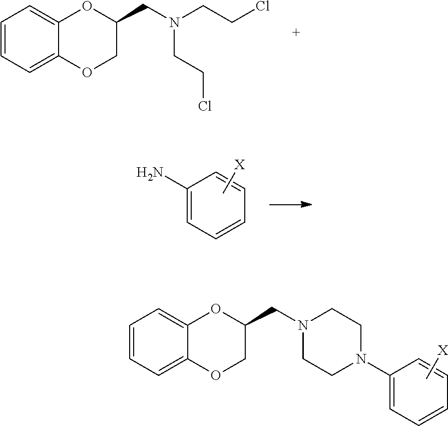

General Procedure: A suitable aniline derivative (0.2 mmol), (S)-2-chloro-N-(2-chloroethyl)-N-((2,3-dihydrobenzo[b]-[1,4]dioxin-2-yl)methyl)ethanamine (0.25 mmol), triethylamine (0.105 ml, 0.75 mmol) and ACN (1 ml) were mixed and heated in a sealed vial at 180° C. for 1-2 h using a microwave reactor. After cooling, the mixture was absorbed on a plug of silica gel. Flash chromatography using a gradient of heptane/EtOAc gave the desired compound.

### Example 26

### (S)-1-((2,3-Dihydrobenzo[b][1,4]dioxin-2-yl)methyl)-4-(2-propylphenyl)piperazine

Using the general procedure, 2-propylaniline was reacted with (S)-2-chloro-N-(2-chloroethyl)-N-((2,3-dihydrobenzo[b][1,4]-dioxin-2-yl)methyl)ethanamine to give 10.4 mg of the title compound.

¹H NMR (CDCl₃): δ 0.97 (t, 3H), 1.59-1.71 (m, 2H), 2.58-2.83 (m, 12H), 3.98-4.08 (m, 1H), 4.33-4.38 (m, 2H), 6.82-6.92 (m, 4H), 7.00-7.23 (m, 4H).

### Example 27

### (S)-1-((2,3-Dihydrobenzo[b][1,4]dioxin-2-yl)methyl)-4-(2-(trifluoromethoxy)phenyl)piperazine

Using the general procedure, 2-(trifluoromethoxy)aniline was reacted with (S)-2-chloro-N-(2-chloroethyl)-N-((2,3-dihydrobenzo[b][1,4]dioxin-2-yl)methyl)ethanamine to give 2.3 mg of the title compound.

¹H NMR (CDCl₃): δ 2.60-2.84 (m, 4H), 3.05-3.16 (m, 4H), 4.00-4.06 (m, 1H), 4.08-4.16 (m, 2H), 4.30-4.37 (m, 2H), 6.82-6.92 (m, 4H), 6.97-7.05 (m, 2H), 7.16-7.25 (m, 2H).

### Example 28

### (S)-1-(Biphenyl-3-yl)-4-((2,3-dihydrobenzo[b][1,4]dioxin-2-yl)methyl)piperazine

Using the general procedure, biphenyl-3-amine was reacted with (S)-2-chloro-N-(2-chloroethyl)-N-((2,3-dihydrobenzo[b][1,4]dioxin-2-yl)methyl)ethanamine to give 12.9 mg of the title compound.

¹H NMR (CDCl₃): δ 2.62-2.88 (m, 6H), 3.23-3.35 (m, 4H), 3.99-4.04 (m, 1H), 4.32-4.40 (m, 2H), 6.78-6.99 (m, 5H), 7.04-7.18 (m, 2H), 7.31-7.50 (m, 4H), 7.56-7.21 (m, 2H).

### Example 29

### (S)-1-((2,3-Dihydrobenzo[b][1,4]dioxin-2-yl)methyl)-4-(2-(furan-2-yl)phenyl)piperazine

Using the general procedure, 2-(furan-2-yl)aniline was reacted with (S)-2-chloro-N-(2-chloroethyl)-N-((2,3-dihydrobenzo[b][1,4]dioxin-2-yl)methyl)ethanamine to give 6.4 mg of the title compound.

¹H NMR (CDCl₃): δ 2.59-2.89 (m, 6H), 2.90-3.08 (m, 4H), 3.99-4.07 (m, 1H), 4.29-4.40 (m, 2H), 6.47-6.52 (m, 1H), 6.80-6.93 (m, 4H), 7.08-7.30 (m, 4H), 7.44-7.49 (m, 1H), 7.77-7.82 (m, 1H).

### Example 30

### (S)-Ethyl 2-(4-((2,3-dihydrobenzo[b][1,4]dioxin-2-yl)methyl)piperazin-1-yl)benzoate

Using the General Procedure, ethyl 2-aminobenzoate was reacted with (S)-2-chloro-N-(2-chloroethyl)-N-((2,3-dihydrobenzo[b][1,4]dioxin-2-yl)methyl)ethanamine to give 5.0 mg of the title compound.

¹H NMR (CDCl₃): δ 1.39 (t, 3H), 2.61-2.83 (m, 6H), 3.01-3.17 (m, 4H), 3.91-4.06 (m, 1H), 4.30-4.40 (m, 4H), 6.81-6.92 (m, 4H), 6.97-7.07 (m, 2H), 7.37-7.44 (m, 1H), 7.69-7.74 (m, 1H).

### Example 31

### (S)-1-((2,3-Dihydrobenzo[b][1,4]dioxin-2-yl)methyl)-4-o-tolylpiperazine

Using the general procedure, o-toluidine was reacted with (S)-2-chloro-N-(2-chloroethyl)-N-((2,3-dihydrobenzo[b][1,4]dioxin-2-yl)methyl)ethanamine to give 27.6 mg of the title compound.

¹H NMR (CDCl₃): δ 2.30 (s, 3H), 2.59-2.85 (m, 6H), 2.85-3.01 (m, 4H), 3.96-4.10 (m, 1H), 4.28-4.42 (m, 2H), 6.79-6.95 (m, 4H), 6.95-7.09 (m, 2H), 7.15-7.22 (m, 2H).

### Example 32

### (S)-1-((2,3-Dihydrobenzo[b][1,4]dioxin-2-yl)methyl)-4-m-tolylpiperazine

Using the general procedure, m-toluidine was reacted with (S)-2-chloro-N-(2-chloroethyl)-N-((2,3-dihydrobenzo[b][1,4]dioxin-2-yl)methyl)ethanamine to give 17.5 mg of the title compound.

¹H NMR (CDCl₃): δ 2.31 (s, 3H), 2.66-2.85 (m, 6H), 3.15-3.28 (m, 4H), 3.97-4.09 (m, 1H), 4.26-4.42 (m, 2H), 6.65-6.80 (m, 3H), 6.80-6.94 (m, 4H), 7.09-7.23 (m, 1H).

### Example 33

### (S)-(3-(4-((2,3-Dihydrobenzo[b][1,4]dioxin-2-yl)methyl)piperazin-1-yl)-4-methylphenyl)methanol

Using the general procedure, (3-amino-4-methylphenyl)methanol was reacted with (S)-2-chloro-N-(2-chloroethyl)-N-((2,3-dihydrobenzo[b][1,4]dioxin-2-yl)methyl)ethanamine to give 5.2 mg of the title compound.

¹H NMR (CDCl₃): δ 2.29 (s, 3H), 2.62-2.85 (m, 6H), 2.92-2.98 (m, 4H), 4.00-4.07 (m, 1H), 4.30-4.39 (m, 2H), 4.64 (s, 2H), 6.80-6.94 (m, 4H), 6.97-7.01 (m, 1H), 7.03-7.06 (m, 1H), 7.14-7.20 (m, 1H).

### Example 34

### (S)-(3-(4-((2,3-Dihydrobenzo[b][1,4]dioxin-2-yl)methyl)piperazin-1-yl)phenyl)methanol

Using the general procedure, (3-aminophenyl)methanol was reacted with (S)-2-chloro-N-(2-chloroethyl)-N-((2,3-dihydrobenzo[b][1,4]dioxin-2-yl)methyl)ethanamine to give 18.9 mg of the title compound.

¹H NMR (CDCl₃): δ 2.57-2.87 (m, 6H), 3.20-3.26 (m, 4H), 3.94-4.00 (m, 1H), 4.24-4.40 (m, 2H), 4.66 (s, 2H), 6.80-6.97 (m, 5H), 6.93-6.97 (m, 1H), 7.18-7.27 (m, 2H).

### Example 35

### (S)-2-(2-(4-((2,3-Dihydrobenzo[b][1,4]dioxin-2-yl)methyl)piperazin-1-yl)phenyl)ethanol

3-(2-Aminophenyl)ethan-1-ol (24 mg, 0.172 mmol), (S)-2-chloro-N-(2-chloroethyl)-N-((2,3-dihydrobenzo[b][1,4]dioxin-2-yl)methyl)ethanamine (50 mg, 0.172 mmol), triethylamine (0.060 ml, 0.43 mmol) and acetonitrile (0.5 ml) were mixed and heated in a sealed vial at 180° C. for 2 h using a microwave reactor. After cooling, the mixture was absorbed on a plug of silica gel. Flash chromatography using a gradient of heptane/EtOAc gave 15.0 mg of the title compound.

¹H NMR (CDCl₃): δ 2.50-3.19 (m, 14H), 3.74-3.19 (m, 2H), 3.98-4.08 (m, 1H), 4.26-4.38 (m, 2H), 4.89 (br s, 1H), 6.75-6.96 (m, 4H), 7.07-7.28 (m, 4H).

### Homopiperazines

### Example 36

### Methyl 2-(4-((2,3-dihydrobenzo[b][1,4]dioxin-2-yl)methyl)-1,4-diazepan-1-yl)benzoate

A mixture of 1-((2,3-dihydrobenzo[b][1,4]dioxin-2-yl)methyl)-1,4-diazepane (0.21 g, 0.86 mmol), methyl 2-fluorobenzoate (0.20 g, 1.28 mmol) and K₂CO₃ (0.18 g, 1.30 mmol) in DMF (8 ml) was heated in a microwave reactor at 220° C. for 30 min. The mixture was poured into water and extracted with EtOAc (3×20 ml). The organic layer was dried and evaporated. The crude product was purified by flash chromatography (gradient of DCM and MeOH) to give 0.20 g of the title compound.

¹H NMR (CDCl₃): δ 1.91 (m, 2H), 2.73 (m, 1H), 2.86 (m, 4H), 3.43 (m, 4H), 3.88 (s, 3H), 3.97 (dd, 1H), 4.33 (m, 2H), 6.85 (m, 1H), 6.81 (m, 5H), 6.97 (d, 1H), 7.31 (t, 1H), 7.59 (dd, 1H).

### Example 37

### (2-(4-((2,3-Dihydrobenzo[b][1,4]dioxin-2-yl)methyl)-1,4-diazepan-1-yl)phenyl)methanol

To a suspension of LAH (100 mg, 2.63 mmol) in dry THF (2 ml) was added methyl 2-(4-((2,3-dihydrobenzo[b][1,4]dioxin-2-yl)methyl)-1,4-diazepan-1-yl)benzoate (200 mg, 0.52 mmol) in dry THF (5 ml). The reaction mixture was heated in a microwave reactor at 80° C. for 10 min. The reaction mixture was poured into ice water and extracted with EtOAc (3×20 ml). The combined organic phases were dried and evaporated. Flash chromatography (heptane/EtOAc, 40:60) of the crude product gave 41 mg of the title compound.

¹H NMR (CDCl₃): δ 1.92 (m, 2H), 2.75 (m, 1H), 2.95 (m, 6H), 3.19 (m, 4H), 4.04 (m, 1H), 4.35 (m, 1H), 4.38 (dd, 1H), 4.78 (s, 2H), 6.89 (m, 4H), 7.07 (t, 1H, 7.18 (m, 3H).

### Example 38

### 2-(4-((2,3-Dihydrobenzo[b][1,4]dioxin-2-yl)methyl)-1,4-diazepan-1-yl)nicotinonitrile

A mixture of 2-(bromomethyl)-2,3-dihydrobenzo[b][1,4]dioxine (150 mg, 0.65 mmol), 2-(1,4-diazepan-1-yl)nicotinonitrile (131 mg, 0.65 mmol) and DIPEA (0.4 ml, 2.32 mmol) in DMF (1.5 ml) was heated in a microwave reactor at 160° C. for 20 min. The mixture was poured into water and extracted with EtOAc (3×20 ml). The combined organic layers were dried and evaporated to dryness. The crude product was purified by flash chromatography (gradient of DCM and MeOH) to give 97 mg of the title compound.

¹H NMR (DMSO-d₆): δ 1.91 (m, 2H), 2.49-2.51 (m, 4H), 2.73 (m, 2H), 3.08-3.92 (m, 5H), 4.23 (m, 2H), 6.72 (dd, 1H), 6.81 (m, 4H), 7.93 (dd, 1H), 8.33 (dd, 1H).

### Example 39

### 2-(4-((2,3-Dihydrobenzo[b][1,4]dioxin-2-yl)methyl)-1,4-diazepan-1-yl)nicotinamide

A mixture of 2-(4-((2,3-dihydrobenzo[b][1,4]dioxin-2-yl)methyl)-1,4-diazepan-1-yl)nicotinonitrile (1.36 g, 3.88 mmol), NaOH (70%, 25 ml) in EtOH (25 ml) was heated at 140° C. for 10 h. The mixture was poured into water and extracted with EtOAc (3×10 ml). The combined organic layers were dried and evaporated to give 0.64 g of the title compound.

¹H NMR (DMSO-d₆): δ 1.84 (m, 2H), 2.63 (m, 5H), 2.87 (m, 2H), 3.51 (t, 2H), 3.59 (t, 2H), 3.92 (dd, 1H), 4.23 (m, 2H), 6.62 (dd, 1H), 6.82 (m, 4H), 7.29 (s, 1H), 7.52 (d, 1H), 7.54 (s, 1H), 8.10 (d, 1H).

### Example 40

### (2-(4-((2,3-Dihydrobenzo[b][1,4]dioxin-2-yl)methyl)-1,4-diazepan-1-yl)pyridin-3-yl)methanol

### Step A: 2-(4-((2,3-Dihydrobenzo[b][1,4]dioxin-2-yl)methyl)-1,4-diazepan-1-yl)nicotinic acid

The aqueous phase from the above experiment was acidified to pH 5 and extracted with EtOAc (3×10 ml). The combined extracts were dried and evaporated to give 0.27 g of the title product.

¹H NMR (DMSO-d₆): δ 1.91 (m, 2H), 2.62 (m, 5H), 2.80 (m, 2H), 3.49 (t, 2H), 3.55 (t, 2H), 3.92 (dd, 1H), 4.25 (m, 2H), 6.73 (dd, 1H), 6.84 (m, 4H), 7.80 (d, 1H), 8.10 (d, 1H).

### Step B: (2-(4-((2,3-Dihydrobenzo[b][1,4]dioxin-2-yl)methyl)-1,4-diazepan-1-yl)pyridin-3-yl)methanol

To a suspension of LAH (0.10 g, 2.63 mmol) in dry THF (3 ml) was added the above obtained nicotinic acid derivative (0.19 g, 0.51 mmol) in dry THF (10 ml). The reaction mixture was heated in a microwave reactor at 80° C. for 10 min. The reaction mixture was poured into ice water and extracted with EtOAc (3×10 ml). The combined organic phases were dried and evaporated. The crude product was purified by flash chromatography (gradient of DCM and MeOH) to give 0.11 g of the title compound.

¹H NMR (CDCl₃): δ 1.96 (m, 2H), 2.78 (m, 2H), 2.93 (m, 6H), 3.45 (m, 5H), 4.04 (m, 1H), 4.33 (m, 1H), 4.36 (m, 1H), 4.68 (s, 1H), 6.83 (m, 4H), 7.55 (d, 1H), 8.20 (d, 1H).

### Example 41

### (S)-(2-(4-((2,3-Dihydrobenzo[b][1,4]dioxin-2-yl)methyl)-1,4-diazepan-1-yl)pyridin-3-yl)methanol

### Step A: (S)-Methyl 2-(4-((2,3-dihydrobenzo[b][1,4]dioxin-2-yl)methyl)-1,4-diazepan-1-yl)nicotinate

(R)-(2,3-Dihydrobenzo[b][1,4]dioxin-2-yl)methyl methanesulfonate (311 mg, 1.27 mmol), methyl 2-(1,4-diazepan-1-yl)nicotinate (300 mg, 1.27 mmol), K₂CO₃ (194 mg, 1.40 mmol) and KI (12 mg) were heated in DMF (9 ml) at 120° C. for 2 h. The cooled mixture was poured into water, which was extracted with EtOAc. The combined extracts were washed several times with water, dried and evaporated. Purification of the crude product by flash chromatography (heptane/EtOAc, 1:1) afforded 71 mg of the title compound.

¹H NMR (CDCl₃): δ 1.92-2.02 (m, 2H), 2.60-2.82 (m, 4H), 2.85-3.01 (m, 2H), 3.48 (t, 2H), 3.65 (t, 2H), 3.86 (s, 3H), 3.95 (dd, 1H), 4.18-4.29 (m, 2H), 6.61 (dd, 1H), 6.78-6.88 (m, 4H), 7.86 (dd, 1H), 8.21 (dd, 1H).

### Step B: (S)-(2-(4-((2,3-Dihydrobenzo[b][1,4]dioxin-2-yl)methyl)-1,4-diazepan-1-yl)pyridin-3-yl)methanol

The ester obtained in the above step (71 mg, 0.19 mmol) was reduced with LAH (28 mg, 0.74 mmol) in dry THF (5 ml, reflux 2 h) to give, after standard work-up with 2.5 M NaOH, 67 mg of the title alcohol.

¹H NMR (CDCl₃): δ 1.87-2.02 (m, 2H), 2.75 (dd, 1H), 2.82-3.01 (m, 5H), 3.43 (dd, 2H), 3.48 (m, 2H), 4.01 (dd, 1H), 4.23-4.31 (m, 1H), 4.33 (dd, 1H), 4.67 (s, 2H), 6.80-6.90 (m, 5H), 7.55 (dd, 1H), 8.18 (dd, 1H).

### Preparation of a Labeled PET Tracer

### Example 42

### (S)-1-((2,3-Dihydrobenzo[b][1,4]dioxin-2-yl)methyl)-4-(3-([¹¹C]-methoxymethyl)pyridin-2-yl)piperazine

(S)-(2-(4-((2,3-Dihydrobenzo[b][1,4]dioxin-2-yl)methyl)piperazin-1-yl)pyridin-3-yl)methanol (1 mg) was methylated with [¹¹C]methyl triflate in ACN (0.2 ml), in the presence 1 M tetrabutylammonium hydroxide (0.003 ml), for 3 minutes at 80° C. Purification with HPLC gave the title compound, suitable for formulation and use as a ¹¹C-labeled PET-tracer.

[¹¹C]methyl triflate was prepared starting from [¹¹C]iodomethane according to the procedure described in Appl. Radiat. Isot. 43 (1992) 1383.

[¹¹C]iodomethane was prepared starting from cyclotrone produced [¹¹C]methane according to the procedure described in Appl. Radiat. Isot. 48 (1997) 153.

As already mentioned hereinbefore, the compounds of formula I show interesting pharmacological properties, namely they exhibit an improved selectivity for the alpha2C adrenoceptor subtype and/or an enhanced potency. Said properties are demonstrated with the pharmacological test presented below.

Experiment 1: Determination of alpha2A and alpha2C Antagonistic Activity In Vitro

Chinese hamster ovary (CHO) cells stably transfected with human alpha2A or alpha2C receptors (University of Turku, Finland) were cotransfected with the expression vector pCEP-Gα16 (Molecular Devices, CA, USA) were used in this experiment. The cells were maintained at 37° C. in a 5% CO₂/95% air atmosphere. The cells were cultured in HAM F-12 medium supplemented with 10% FCS, 25 mM HEPES, 100 IU/ml penicillin, 100 μg/ml streptomycin, 500 μg/ml geneticin and 240 μg/ml hygromycin B. The cells were subcultured twice weekly with 0.25% trypsin and 1 mM EDTA. The subculture ratio was 1:5-1:20. The growth medium was changed every 2 or 3 days. All cell culture reagents were from Gibco. The day before the experiment the cells were plated into black-walled, clear bottom 96-well plates at a density of 30,000-45,000 cells/well.

The growth medium was removed and the cells were incubated with the test compounds and the FLIPR Calcium 3 Assay reagent (Molecular Devices, CA, USA) for 1 h at 37° C. in dark. The test compounds (concentrations in cells 100 pM-10 μM) were dissolved in Probenecid-Ringer consisting of 150 mM NaCl, 3 mM KCl, 1.2 mM MgCl₂, 1 mM CaCl₂, 5 mM glucose, 20 mM HEPES and 2.5 mM probenecid (pH 7.4 adjusted with 1.0 M NaOH). The osmolarity was adjusted to 322 milliosmoles with Osmostat® OM-6020 osmometer (DIC Kyoto Daiichi Kagagu Co. Ltd, Japan). The changes in intracellular calcium were monitored using FLEXstation benchtop scanning fluorometer with integrated fluid transfer workstation (Molecular Devices, CA, USA) and displayed using SOFTmax PRO version 3.2 software. All experiments were performed at 37° C. The test compounds dissolved in Probenecid-Ringer were applied by FLEX station at 17 s time point. The IC₅₀ value for a given test compound was determined from dose-response curves, which ranged from 0.01 nM to 10 μM. In order to determine antagonism, the cells were stimulated either with 100 nM adrenaline or 200 nM noradrenaline and the test compounds were added to the cells at least 5 min before the experiment. Typically, there were four replicates at each concentration and seven different dose levels. For example, if the number of plates from which results were obtained was three, 84 (4*7*3) wells were thus measured to construct dose-response relationship. The samples were excited at 485 nm and emission was detected at 525 nm with a 515 nm cut-off filter. Reading time was 60 s per well and the photomultiplier sensitivity value was set to 15. The minimum fluorescence value subtracted from the maximum value for each well was used in the calculations. SOFTmax PRO version 3.2 software was used for analyzing the results. Fitting of the antagonist dose-response results was performed with the free Hill equation and the IC₅₀ values were fitted with Michaelis-Menten equation in Sigma Plot 8.0.

The results are shown in Table 1.

TABLE 1
Alpha2A and alpha2C antagonistic activity in vitro.

IC₅₀/nM

| Compound | Alpha2A | Alpha2C |
|---|---|---|
| Compound of example 17 | 2175 | 8.2 |
| Compound of example 36 | 3900 | 13.7 |
| Compound of example 31 | 16929 | 17.6 |
| Compound of example 29 | 12703 | 18.0 |
| Compound of example 23 | 10741 | 30.6 |
| Compound of example 18 | 41061 | 134.4 |

In vivo effects of the compounds of formula I can be demonstrated with the pharmacological tests as described in WO 03/082866.

The compounds of formula I exhibit alpha2C antagonistic activity. The present invention thus provides compounds for use as a medicament. Compounds for use in the treatment of diseases or conditions where an alpha2C antagonist is indicated to be useful are also provided. Furthermore, a method for the treatment of diseases or conditions where an alpha2C antagonist is indicated to be useful is provided. In said method an effective amount of at least one compound of formula I is administered to a mammal, e.g. human, in need of such treatment. The use of the compounds of formula I for the manufacture of a medicament for the treatment of diseases or conditions where an alpha2C antagonist is indicated to be useful is also provided.

In one embodiment of the invention the aforementioned disease or condition where an alpha2C antagonist is indicated to be useful is a mental disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease or mild cognitive impairment; for example, a mental disorder propagated by stress, Parkinson's disease, depression, schizophrenia, attention deficit hyperactivity disorder, obsessive compulsive disorder or Alzheimer's disease; such as a mental disorder propagated by stress, depression or schizophrenia.

Representative examples of drug-induced psychoses include, but are not limited to, psychosis caused by chronic use of dopaminergic agents.

Representative examples of disorders caused by fluctuation of the levels of sex hormones include, but are not limited to, premenstrual syndrome and hot flashes.

The compounds of the invention can be administered, for example, enterally, topically or parenterally by means of any pharmaceutical formulation useful for said administration and comprising at least one active compound of formula I in pharmaceutically acceptable and effective amounts together with pharmaceutically acceptable diluents, carriers and/or excipients known in the art. The manufacture of such pharmaceutical formulations is known in the art.

The therapeutic dose to be given to a subject in need of the treatment will vary depending on the compound being administered, the species, the age and the sex of the subject being treated, the particular condition being treated, as well as the route and method of administration, and is easily determined by a person skilled in the art. Accordingly, the typical dosage for oral administration is from 10 ng/kg to 100 mg/kg per day and for parenteral administration from 1 ng/kg to 10 mg/kg for an adult mammal.

The compounds of the invention are given to the subject as such or in combination with one or more other active ingredients, each in its own composition or some or all of the active ingredients combined in a single composition, and/or suitable pharmaceutical excipients. Suitable pharmaceutical excipients include conventionally used excipients and formulation aids, such as fillers, binders, disintegrating agents, lubricants, solvents, gel forming agents, emulsifiers, stabilizers, colorants and/or preservatives.

The compounds of the invention are formulated into dosage forms using commonly known pharmaceutical manufacturing methods. The dosage forms can be, for example, tablets, capsules, granules, suppositories, emulsions, suspensions or solutions. Depending on the route of administration and the galenic form, the amount of the active ingredient in a formulation can typically vary between 0.01% and 100% by weight.

A person skilled in the art will appreciate that the embodiments described in this application can be modified without departing from the inventive concept. A person skilled in the art also understands that the invention is not limited to the particular embodiments disclosed but is intended to also cover modifications of the embodiments that are within the scope of the invention.

1. A compound of formula I, 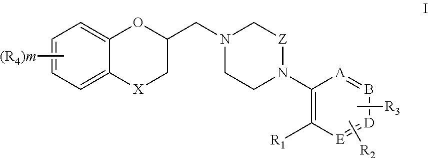
wherein
X is O, S or CH₂;
Z is —[CH₂]ₙ—;
A, B, D and E are independently C or N provided that at least three of A, B, D and E are C;
R₁ is H, halogen, hydroxy, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, hydroxy(C₁-C₆)alkoxy(C₁-C₆)alkyl, (C₁-C₆)alkoxy(C₁-C₆)alkoxy(C₁-C₆)alkyl, (C₁-C₆)alkoxy-(C═O)—, CN, (R₅)₂N—, (R₅)₂N—(C₁-C₆)alkyl, (R₅)₂N—(C═O)—, SH—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, hydroxy(C₁-C₆)alkyl-S(Op)-(C₁-C₆)alkyl, (C₁-C₆)alkoxy(C₁-C₆)alkyl-S(Op)-(C₁-C₆)alkyl or furyl;
R₂ is H, halogen, (C₁-C₆)alkyl, (C₁-C₆)alkoxy or hydroxy(C₁-C₆)alkyl;
R₃ is H, halogen, (C₁-C₆)alkyl or phenyl;
R₄ is halogen, hydroxy, (C₁-C₆)alkyl, (C₁-C₆)alkoxy, CN or (R₅)₂N—;
R₅ is, independently at each occurrence, H, (C₁-C₆)alkyl or (C₁-C₆)alkoxy(C₁-C₆)alkyl;
m is 0, 1 or 2;
n is 1 or 2; and
p is 1 or 2,
where compound I is present in its labeled or unlabeled form,
or a pharmaceutically acceptable salt or ester thereof, with the provisos, that
a) R₁, R₂ and R₃ are not simultaneously H;
b) when A is C and two of R₁, R₂ and R₃ is H, then the third of R₁, R₂ and R₃ is not halogen;
c) the compound is not 1-((2,3-dihydrobenzo[b][1,4]dioxin-2-yl)methyl)-4-(2-methoxyphenyl)piperazine, 1-(chroman-2-ylmethyl)-4-o-tolylpiperazine or 1-((2,3-dihydrobenzo[b][1,4]dioxin-2-yl)methyl)-4-(6-methylpyridin-2-yl)piperazine.

2. The compound according to claim 1, wherein X is O.

3. The compound according to claim 1, wherein A, B, D and E are C.

4. The compound according to claim 1, wherein A is N; and B, D and E are C.

5. The compound according to claim 1, wherein n is 1.

6. The compound according to claim 1, wherein n is 2.

7. The compound according to claim 1, wherein
X is O, S or CH₂;
Z is —[CH₂]ₙ—;
A is C or N;
B, D and E are C;
R₁ is H, halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurrence, H or (C₁-C₆)alkyl;
m is 0; and
n is 1 or 2.

8. The compound according to claim 7, wherein
X is O;
Z is —[CH₂]ₙ—;
A is C or N;
B, D and E are C;
R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurrence, H or (C₁-C₆)alkyl;
m is 0; and
n is 1 or 2.

9. The compound according to claim 8, wherein
X is O;
Z is —[CH₂]ₙ—;
A, B, D and E are C;
R₁ is (C₁-C₆)alkyl, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H or halogen;
R₃ is H;
R₅ is, independently at each occurrence, H or (C₁-C₆)alkyl;
m is 0; and
n is 1 or 2.

10. The compound according to claim 8, wherein
X is O;
Z is —[CH₂]ₙ—;
A is N;
B, D and E are C;
R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H or halogen;
R₃ is H;
R₅ is, independently at each occurrence, H or (C₁-C₆)alkyl;
m is 0; and
n is 1 or 2.

11. The compound according to claim 8, wherein
X is O;
Z is —[CH₂]ₙ—;
A is N;
B, D and E are C;
R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurrence, H or (C₁-C₆)alkyl;
m is 0; and
n is 1.

12. The compound according to claim 8, wherein
X is O;
Z is —[CH₂]ₙ—;
A is N;
B, D and E are C;
R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurrence, H or (C₁-C₆)alkyl;
m is 0; and
n is 2.

13. The compound according to claim 8, wherein
X is O;
Z is —[CH₂]ₙ—;
A, B, D and E are C;
R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurrence, H or (C₁-C₆)alkyl;
m is 0; and
n is 1.

14. The compound according to claim 8, wherein
X is O;
Z is —[CH₂]ₙ—;
A, B, D and E are C;
R₁ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N—(C₁-C₆)alkyl, (R₅)₂N—(C═O)— or furyl;
R₂ is H, halogen, (C₁-C₆)alkyl or hydroxy(C₁-C₆)alkyl;
R₃ is H, (C₁-C₆)alkyl or phenyl;
R₅ is, independently at each occurrence, H or (C₁-C₆)alkyl;
m is 0; and
n is 2.

15. The compound according to claim 1, wherein the compound is methyl 2-(4-((2,3-dihydrobenzo[b][1,4]dioxin-2-yl)methyl)piperazin-1-yl)benzoate, (2-(4-((2,3-dihydrobenzo[b][1,4]dioxin-2-yl)methyl)piperazin-1-yl)phenyl)methanol, 1-((2,3-dihydrobenzo[b][1,4]dioxin-2-yl)methyl)-4-(2-(methoxymethyl)phenyl)piperazine, 2-(4-((2,3-dihydrobenzo[b][1,4]dioxin-2-yl)methyl)piperazin-1-yl)benzonitrile, (2-(4-((2,3-dihydrobenzo[b][1,4]dioxin-2-yl)methyl)piperazin-1-yl)phenyl)methanamine, 1-(2-(4-((2,3-dihydrobenzo[b][1,4]dioxin-2-yl)methyl)piperazin-1-yl)phenyl)-N-methylmethanamine, 1-((2,3-dihydrobenzo[b][1,4]dioxin-2-yl)methyl)-4-(2-(ethoxymethyl)phenyl)piperazine, 2-(2-(4-((2,3-dihydrobenzo[b][1,4]dioxin-2-yl)methyl)piperazin-1-yl)phenyl)propan-2-ol, 1-((2,3-dihydrobenzo[b][1,4]dioxin-2-yl)methyl)-4-(3-(methoxymethyl)pyridin-2-yl)piperazine, (S)-(2-(4-((7-fluoro-2,3-dihydrobenzo[b][1,4]dioxin-2-yl)methyl)-piperazin-1-yl)pyridin-3-yl)methanol, (S)-(2-(4-((7-fluoro-2,3-dihydrobenzo[b][1,4]dioxin-2-yl)methyl)-piperazin-1-yl)pyridin-3-yl)methanol≅HCl, (S)-1-((7-fluoro-2,3-dihydrobenzo[b][1,4]dioxin-2-yl)methyl)-4-(3-(methoxymethyl)pyridin-2-yl)piperazine≅HCl, (S)-1-((2,3-dihydrobenzo[b][1,4]dioxin-2-yl)methyl)-4-(3-((2-fluoroethoxy)methyl)pyridin-2-yl)piperazine, 1-(2,3-dichlorophenyl)-4-((2,3-dihydrobenzo[b][1,4]dioxin-2-yl)methyl)piperazine, (2-(4-((2,3-dihydrobenzo[b][1,4]dioxin-2-yl)methyl)piperazin-1-yl)pyridin-3-yl)methanol, (S)-(2-(4-((2,3-dihydrobenzo[b][1,4]dioxin-2-yl)methyl)piperazin-1-yl)pyridin-3-yl)methanol, (S)-1-((2,3-dihydrobenzo[b][1,4]dioxin-2-yl)methyl)-4-(2-(methoxymethyl)phenyl)piperazine, (R)-1-((2,3-dihydrobenzo[b][1,4]dioxin-2-yl)methyl)-4-(2-(methoxymethyl)phenyl)piperazine, (S)-(2-(4-((2,3-dihydrobenzo[b][1,4]dioxin-2-yl)methyl)piperazin-1-yl)phenyl)methanol, (S)-1-((2,3-dihydrobenzo[b][1,4]dioxin-2-yl)methyl)-4-(3-(methoxymethyl)pyridin-2-yl)piperazine, (1-((2,3-dihydrobenzo[b][1,4]oxathiin-2-yl)methyl)-4-(2-(methoxymethyl)phenyl)piperazine, 1-(chroman-2-ylmethyl)-4-(2-(methoxymethyl)phenyl)piperazine, (2-(4-((2,3-dihydrobenzo[b][1,4]dioxin-2-yl)methyl)piperazin-1-yl)-6-fluorophenyl)methanol, (2-(4-((2,3-dihydrobenzo[b][1,4]dioxin-2-yl)methy)piperazin-1-yl)-3-fluorophenyl)methanol, (2-(4-((2,3-dihydrobenzo[b][1,4]dioxin-2-yl)methyl)piperazin-1-yl)-5-fluorophenyl)methanol, (S)-1-((2,3-dihydrobenzo[b][1,4]dioxin-2-yl)methyl)-4-(2-propylphenyl)piperazine, (S)-1-((2,3-dihydrobenzo[b][1,4]dioxin-2-yl)methyl)-4-(2-(trifluoromethoxy)phenyl)piperazine, (S)-1-(biphenyl-3-yl)-4-((2,3-dihydrobenzo[b][1,4]dioxin-2-yl)methyl)piperazine, (S)-1-((2,3-dihydrobenzo[b][1,4]dioxin-2-yl)methyl)-4-(2-(furan-2-yl)phenyl)piperazine, (S)-ethyl 2-(4-((2,3-dihydrobenzo[b][1,4]dioxin-2-yl)methyl)piperazin-1-yl)benzoate, (S)-1-((2,3-dihydrobenzo[b][1,4]dioxin-2-yl)methyl)-4-o-tolylpiperazine, (S)-1-((2,3-dihydrobenzo[b][1,4]dioxin-2-yl)methyl)-4-m-tolylpiperazine, (S)-(3-(4-((2,3-dihydrobenzo[b][1,4]dioxin-2-yl)methyl)piperazin-1-yl)-4-methylphenyl)methanol, (S)-(3-(4-((2,3-dihydrobenzo[b][1,4]dioxin-2-yl)methyl)piperazin-1-yl)phenyl)methanol, (S)-2-(2-(4-((2,3-dihydrobenzo[b][1,4]dioxin-2-yl)methyl)piperazin-1-yl)phenyl)ethanol, methyl 2-(4-((2,3-dihydrobenzo[b][1,4]dioxin-2-yl)methyl)-1,4-diazepan-1-yl)benzoate, (2-(4-((2,3-dihydrobenzo[b][1,4]dioxin-2-yl)methyl)-1,4-diazepan-1-yl)phenyl)methanol, 2-(4-((2,3-dihydrobenzo[b][1,4]dioxin-2-yl)methyl)-1,4-diazepan-1-yl)nicotinonitrile, 2-(4-((2,3-dihydrobenzo[b][1,4]dioxin-2-yl)methyl)-1,4-diazepan-1-yl)nicotinamide, (2-(4-((2,3-dihydrobenzo[b][1,4]dioxin-2-yl)methyl)-1,4-diazepan-1-yl)pyridin-3-yl)methanol or (S)-(2-(4-((2,3-dihydrobenzo[b][1,4]dioxin-2-yl)methyl)-1,4-diazepan-1-yl)pyridin-3-yl)methanol.

16. The compound according to claim 1, wherein the compound of formula 1 is carbon-isotope labeled.

17. The compound according to claim 16, wherein the compound is (S)-1-((2,3-dihydrobenzo[b][1,4]dioxin-2-yl)methyl)-4-(3-([¹¹C]-methoxymethyl)pyridin-2-yl)piperazine.

18-20. (canceled)

21. A method for the treatment of a disease or a condition where an alpha2C antagonist is indicated to be useful, which method comprises administering to a mammal in need of such treatment an effective amount of at least one compound of formula I, 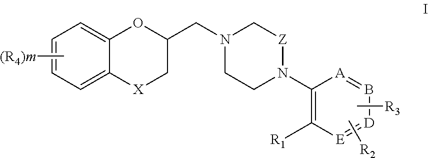
wherein
X is O, S or CH₂;
Z is —[CH₂]ₙ—;
A, B, D and E are independently C or N provided that at least three of A, B, D and E are C;
R₁ is H, halogen, hydroxy, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, hydroxy(C₁-C₆)alkoxy(C₁-C₆)alkyl, (C₁-C₆)alkoxy(C₁-C₆)alkoxy(C₁-C₆)alkyl, (C₁-C₆)alkoxy-(C═O)—, CN, (R₅)₂N—, (R₅)₂N—(C₁-C₆)alkyl, (R₅)₂N—(C═O)—, SH—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, hydroxy(C₁-C₆)alkyl-S(Op)-(C₁-C₆)alkyl, (C₁-C₆)alkoxy(C₁-C₆)alkyl-S(Op)-(C₁-C₆)alkyl or furyl;
R₂ is H, halogen, (C₁-C₆)alkyl, (C₁-C₆)alkoxy or hydroxy(C₁-C₆)alkyl;
R₃ is H, halogen, (C₁-C₆)alkyl or phenyl;
R₄ is halogen, hydroxy, (C₁-C₆)alkyl, (C₁-C₆)alkoxy, CN or (R₅)₂N—;
R₅ is, independently at each occurrence, H, (C₁-C₆)alkyl or (C₁-C₆)alkoxy(C₁-C₆)alkyl;
m is 0, 1 or 2;
n is 1 or 2; and
p is 1 or 2,
where compound I is present in its labeled or unlabeled form,
or a pharmaceutically acceptable salt or ester thereof, with the provisos, that
a) R₁, R₂ and R₃ are not simultaneously H;
b) when A is C and two of R₁, R₂ and R₃ is H then the third of R₁, R₂ and R₃ is not halogen;
c) the compound is not 1-((2,3-dihydrobenzo[b][1,4]dioxin-2-yl)methyl)-4-(2-methoxyphenyl)piperazine, 1-(chroman-2-ylmethyl)-4-o-tolylpiperazine or 1-((2,3-dihydrobenzo[b][1,4]dioxin-2-yl)methyl)-4-(6-methylpyridin-2-yl)piperazine.

22. The method according to claim 21, wherein the disease or the condition is a mental disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease or mild cognitive impairment.

23. A pharmaceutical composition comprising at least one compound according to claim 1 and a pharmaceutically acceptable carrier, diluent and/or excipient.

24. The pharmaceutical composition according to claim 23 wherein the composition comprises further at least one other active ingredient.

25-27. (canceled)

28. A method for quantifying alpha2C adrenoceptor binding affinity in vivo comprising
administering a compound according to claim 16 to an animal or human;
measuring the amount of compound according to claim 16 that binds to alpha2C adenoreceptors using positron emission tomography (PET).

29. The method according to claim 28, wherein the compound is (S)-1-((2,3-dihydrobenzo[b][1,4]dioxin-2-yl)methyl)-4-(3-([¹¹C]-methoxymethyl)pyridin-2-yl)piperazine.
